(19) United States
(12) Patent Application Publication
(10) Pub. No.: US 2017/0333889 A1
(43) Pub. Date: Nov. 23, 2017

(54) METHODS FOR DEHYDROGENATING ONE OR MORE ALKANES

(71) Applicant: Flint Hills Resources, LP, Wichita, KS (US)

(72) Inventors: Brian Clancy-Jundt, League City, TX (US); Jan Peeters, Houston, TX (US)

(73) Assignee: Flint Hills Resources, LP, Wichita, KS (US)

(21) Appl. No.: 15/522,551

(22) PCT Filed: Oct. 29, 2015
(86) PCT No.: PCT/US2015/058107

§ 371 Date: Apr. 27, 2017

Related U.S. Application Data
(60) Provisional application No. 62/072,391, filed on Oct. 29, 2014.

(51) Int. Cl. B01J 38/18; C07C 5/333

(52) U.S. Cl. CPC B01J 38/18; C07C 5/3335; C07C 5/3337

(57) ABSTRACT

Methods for dehydrogenating one or more alkanes. A catalyst can be contacted with an alkane under a pressure of less than 101 kPa to produce a coked catalyst and a dehydrogenated product. The dehydrogenated product can be separated from the coked catalyst and the coked catalyst can be contacted with a purge fluid to remove at least a portion of any residual alkane, any residual alkene, or a combination thereof from the coked catalyst. The coked catalyst can be contacted with an oxygen-containing fluid and at least a portion of the coke disposed on the catalyst can be combusted in the presence of the oxygen-containing fluid to produce a decoked catalyst. The decoked catalyst can be contacted with a reducing gas to produce a regenerated catalyst and an off-gas. Additional alkane can be contacted with the regenerated catalyst to produce additional dehydrogenated product and additional coked catalyst.

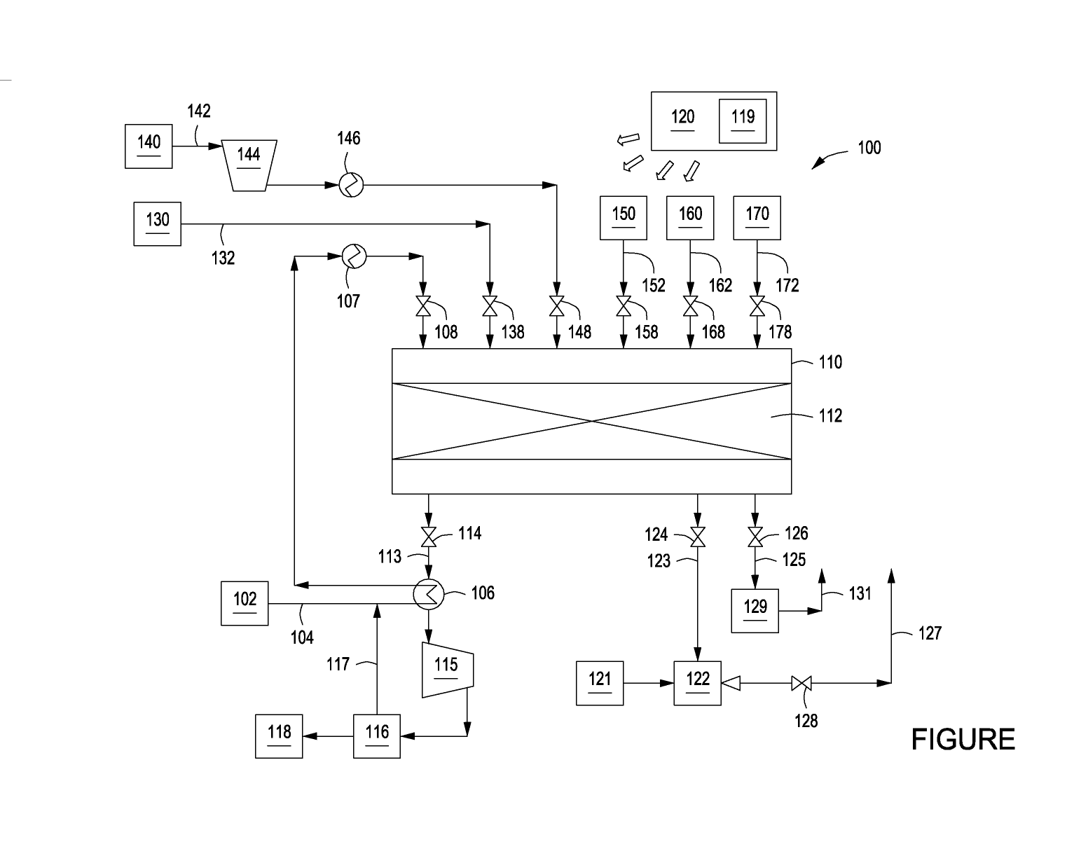

### CROSS-REFERENCE TO RELATED APPLICATIONS

This is a National Stage application under 35 U.S.C. §371 of PCT/US2015/058107, filed on Oct. 29, 2015, and published as WO 2016/069918, which claims priority to U.S. Provisional Patent Application No. 62/072,391, filed on Oct. 29, 2014, which are incorporated by reference herein.

### BACKGROUND

### Field

Embodiments described generally relate to methods for dehydrogenating one or more alkanes.

### Description of the Related Art

The allylic carbon in propylene makes propylene a useful compound for the production of numerous products, e.g., polypropylene, acrylonitrile, propylene oxide, propylene glycol, and cumene. These products can be used as final products and/or as intermediate products to produce other products. One process for producing propylene includes the catalytic dehydrogenation of propane that typically uses a noble-metal catalyst or a heavy-metal catalyst such as platinum or chromium on an acidic support such as alumina or zeolitic materials. The catalytic dehydrogenation of propane to propylene is endothermic and requires a relatively high temperature for the reaction to proceed at a satisfactory rate.

The catalytic dehydrogenation of propane to propylene, however, also needs to be controlled to limit the conversion of propane to methane and ethylene as well as the hydrogenation of ethylene to ethane from hydrogen produced by dehydrogenating the propane. The dehydrogenation of propane also leads to coking of the catalyst, which deactivates the catalyst. As such, the catalyst needs to be regenerated on a regular basis after relatively short periods of operation or residence time in the dehydrogenation reactor.

There is a need, therefore, for improved methods for the catalytic dehydrogenation of alkanes to alkenes, such as propane to propylene or butane to butylene.

### SUMMARY

Methods for dehydrogenating one or more alkanes to produce one or more alkenes are provided. In at least one specific embodiment, the method can include contacting a catalyst with an alkane to produce a coked catalyst that can include coke disposed thereon and a dehydrogenated product that can include an alkene. The alkane can be contacted with the catalyst under a pressure of less than 101 kPa. The dehydrogenated product can be separated from the coked catalyst and the coked catalyst can be contacted with a purge fluid to remove at least a portion of any residual alkane, any residual alkene, or a combination thereof from the coked catalyst. The coked catalyst can be contacted with an oxygen-containing fluid. At least a portion of the coke disposed on the catalyst can be combusted in the presence of the oxygen-containing fluid to produce a decoked catalyst. The decoked catalyst can be contacted with a reducing gas to produce a regenerated catalyst and an off-gas. Additional alkane can be contacted with the regenerated catalyst to produce additional dehydrogenated product and additional coked catalyst.

In at least one other specific embodiment, the method can include introducing an alkane to a reactor that can include a catalyst disposed therein. The catalyst can be contacted with the alkane to produce a coked catalyst that can include coke disposed thereon and a dehydrogenated product that can include an alkene. The alkane can be contacted with the catalyst under a pressure of less than 101 kPa. The dehydrogenated product can be removed from the reactor. The coked catalyst can be contacted with a purge fluid to remove at least a portion of any residual alkane, any residual alkene, or a combination thereof from the coked catalyst. The coked catalyst can be contacted with an oxygen-containing fluid heated to a temperature of at least 600° C. to about 800° C. At least a portion of the coke disposed on the catalyst can be combusted in the presence of the oxygen-containing fluid to produce a decoked catalyst. The decoked catalyst can be contacted with a reducing gas to produce a regenerated catalyst. Additional alkane can be introduced to the reactor that includes the regenerated catalyst. The contact of the decoked catalyst with the reducing gas can be stopped at least 10 seconds prior to introduction of the additional alkane to the reactor. The additional alkane can be contacted with the regenerated catalyst to produce additional dehydrogenated product and additional coked catalyst.

In at least one other specific embodiment, the method can include contacting a catalyst with propane within a process environment to produce propylene and a coked catalyst that can include coke disposed thereon. The process environment can be at a pressure of about 20 kPa to about 38 kPa when the propane is contacted with the regenerated catalyst. The propane can be at a temperature of about 400° C. to about 650° C. when the propane is contacted with the regenerated catalyst. The regenerated catalyst can be at a temperature of about 425° C. to about 750° C. when the propane is contacted with the regenerated catalyst. At least a portion of the propylene can be removed from the process environment. The coked catalyst can be contacted with a purge fluid to remove at least a portion of any residual propane, at least a portion of any residual propylene, or both from the process environment that can include the coked catalyst. The purge fluid can be at a temperature of about 120° C. to about 600° C. The coked catalyst can be contacted with an oxygen-containing fluid within the process environment to combust at least a portion of the coke disposed thereon and produce a decoked catalyst. The oxygen-containing fluid can be at a temperature of about 500° C. to about 800° C. when the coked catalyst is contacted with the oxygen-containing fluid. The decoked catalyst can be contacted with a reducing gas within the process environment to produce a regenerated catalyst. Contact of the reducing gas with the regenerated catalyst within the process environment can be stopped. At least a portion of any residual reducing gas can be removed from the process environment that can include the regenerated catalyst by exposing the process environment to a vacuum for at least 10 seconds after stopping contact of the reducing gas with the regenerated catalyst. Additional propane can be contacted with the regenerated catalyst within the process environment to produce additional propylene and additional coked catalyst that can include coke disposed thereon.

### BRIEF DESCRIPTION OF THE DRAWINGS

The FIGURE depicts a schematic of an illustrative system for dehydrogenating one or more alkanes to produce a dehydrogenated product, according to one or more embodiments described.

### DETAILED DESCRIPTION

One or more alkanes can be contacted with one or more catalysts to produce a dehydrogenated product that includes one or more alkenes and a coked catalyst. For example, the alkane can be introduced into a reactor or other process environment and contacted with the catalyst within the reactor or other process environment under conditions sufficient to produce the dehydrogenated product and the coked catalyst. The dehydrogenation of the alkane is an endothermic reaction. Accordingly, the alkane and/or the catalyst can be heated to a temperature sufficient to provide at least a portion of the heat required to dehydrogenate the alkane in the presence of the catalyst. The catalyst can be a fresh or new catalyst, a regenerated catalyst, or a mixture thereof.

The catalyst can be at a temperature of about 425° C., about 450° C., about 475° C., about 500° C., about 535° C., about 550° C., about 565° C., about 585° C., or about 590° C. to about 595° C., about 600° C., about 625° C., about 650° C., about 675° C., about 700° C., about 750° C., or about 800° C. at the time the alkane is initially introduced into the reactor. For example, the catalyst can be at a temperature of about 440° C. to about 510° C., about 490° C. to about 540° C., about 535° C. to about 700° C., about 560° C. to about 640° C., or about 580° C. to about 610° C. at the time the alkane is initially introduced into the reactor. The alkane can be at a temperature of about 400° C., about 425° C., about 450° C., about 475° C. or about 500° C. to about 525° C., about 550° C., about 575° C., about 600° C., about 625° C., or about 650° C. at the time the alkane is initially introduced into the reactor. For example, the alkane can be at a temperature of about 425° C. to about 648° C., about 490° C. to about 540° C., or about 520° C. to about 620° C. at the time the alkane is initially introduced into the reactor.

The catalyst can be at a temperature of about 425° C., about 450° C., about 475° C., about 500° C., about 535° C., about 550° C., about 565° C., about 585° C. or about 590° C. to about 595° C., about 600° C., about 625° C., about 650° C., about 675° C., about 700° C., about 750° C., or about 800° C. when contacted with the alkane. For example, the catalyst can be at a temperature of about 440° C. to about 510° C., about 490° C. to about 540° C., about 535° C. to about 700° C., about 560° C. to about 640° C., or about 580° C. to about 610° C. when contacted with the alkane. The alkane can be at a temperature of about 400° C., about 425° C., about 450° C., about 475° C. or about 500° C. to about 525° C., about 550° C., about 575° C., about 600° C., about 625° C., or about 650° C. when contacted with the catalyst. For example, the alkane can be at a temperature of about 425° C. to about 648° C., about 490° C. to about 540° C., or about 520° C. to about 620° C. when contacted with the catalyst.

Illustrative alkanes can be or include, but are not limited to, ethane, propane, butane, 2-methylpropane, pentane, 2-methylbutane, 2,2-dimethylpropane, isomers thereof, or any mixture thereof. The dehydrogenated product can be or include, but is not limited to, ethene (ethylene), propene (propylene), but-1-ene (butylene), (Z)-but-2-ene, (E)-but-2-ene, 2-methylpropene, 1-pentene, cis-2-pentene, trans-2-pentene, isomers thereof, or any mixture thereof. In at least one embodiment, the alkane can be or include propane and the dehydrogenated product can be or include propylene. For example, the alkane can include at least 80 wt % of propane, at least 85 wt % of propane, at least 90 wt % of propane, at least 95 wt % of propane, at least 97 wt % of propane, at least 98 wt % of propane, at least 99 wt % of propane, at least 99.5 wt % of propane, at least 99.7 wt % of propane, or at least 99.9 wt % of propane. In other examples, the alkane can include at least 80 wt % of butane, at least 85 wt % of butane, at least 90 wt % of butane, at least 95 wt % of butane, at least 97 wt % of butane, at least 98 wt % of butane, at least 99 wt % of butane, at least 99.5 wt % of butane, at least 99.7 wt % of butane, or at least 99.9 wt % of butane. The dehydrogenation of the alkane to the alkene can be incomplete. As such, the dehydrogenated product can include the dehydrogenated product and the alkane that was not dehydrogenated. In one or more examples, at least a portion of the alkane present in the dehydrogenated product can be separated therefrom and recycled or combined with fresh or incoming alkane and contacted with the catalyst.

The catalyst can include one or more metals. The one or more metals can include, but are not limited to, platinum, palladium, gold, indium, chromium, tin, iron, molybdenum, tungsten, nickel, silver, any alloy thereof, any oxide thereof, or any mixture thereof. The one or more metals can be supported or unsupported. Illustrative supports can include, but are not limited to, one or more inorganic oxides. Illustrative inorganic oxides can include, but are not limited to, alumina, silica, titania, zirconia, boria, zinc oxide, magnesia, or any mixture thereof. Illustrative combinations of inorganic oxides can include, but are not limited to, alumina-silica, silica-titania, alumina-silica-titania, alumina-zirconia, alumina-titania, or any mixture thereof. In at least one example, the support can be or include alumina, silica, or a combination thereof. The catalyst can be disposed within the reactor within a fixed bed and the alkane can be introduced to a first side of the fixed bed and the dehydrogenated product can be recovered from a second side of the fixed bed. In at least one example, the first side and the second side of the fixed bed can be on opposite sides of the fixed bed with respect to one another.

The pressure within the reactor can be less than atmospheric pressure when the alkane is initially introduced into the reactor. For example, the pressure within the reactor or other process environment, when the alkane is initially introduced into the reactor, can be less than 101 kPa, less than 90 kPa, less than 70 kPa, less than 50 kPa, less than 45 kPa, less than 40 kPa, less than 38 kPa, less than 37 kPa, less than 36 kPa, less than 35 kPa, less than 34 kPa, less than 33 kPa, less than 32 kPa, less than less than 31 kPa, less than 30 kPa, less than 29 kPa, less than 28 kPa, less than 27 kPa, less than 26 kPa, less than 25 kPa, less than 24 kPa, less than 23 kPa, less than 22 kPa, or less than 21 kPa. For example, the reactor can be at a pressure a pressure of less than 37.9 kPa, less than 36.5 kPa, less than 34.5 kPa, less than 33.8 kPa, less than 33.1 kPa, or less than 32.4 kPa when the alkane is initially introduced into the reactor. In another example, the reactor can be at a pressure of about 20 kPa to about 38 kPa, about 25 kPa to about 40 kPa, about 27 kPa to about 38 kPa, about 30 kPa to about 37 kPa, about 28 kPa to about 36 kPa, about 28.5 kPa to about 34.5 kPa, about 20 kPa to about 30 kPa, about 20 to about 29, about 20 to about 28, about 22 to about 27, about 28 to about 32, or about 31 kPa to about 33 kPa when the alkane is initially introduced into the reactor.

The alkane can be contacted with the catalyst under a pressure of less than atmospheric pressure. For example, the alkane can be contacted with the catalyst under a pressure of less than 101 kPa, less than 90 kPa, less than 70 kPa, less than 50 kPa, less than 45 kPa, less than 40 kPa, less than 38 kPa, less than 37 kPa, less than 36 kPa, less than 35 kPa, less than 34 kPa, less than 33 kPa, less than 32 kPa, less than less than 31 kPa, less than 30 kPa, less than 29 kPa, less than 28 kPa, less than 27 kPa, less than 26 kPa, less than 25 kPa, less than 24 kPa, less than 23 kPa, less than 22 kPa, or less than 21 kPa. For example, the alkane can be contacted with the catalyst under a pressure of less than 37.9 kPa, less than 36.5 kPa, less than 34.5 kPa, less than 33.8 kPa, less than 33.1 kPa, or less than 32.4 kPa. In another example, the alkane can be contacted with the catalyst under a pressure of about 20 kPa to about 38 kPa, about 25 kPa to about 40 kPa, about 27 kPa to about 38 kPa, about 30 kPa to about 37 kPa, about 28 kPa to about 36 kPa, about 28.5 kPa to about 34.5 kPa, about 20 kPa to about 30 kPa, about 20 to about 29, about 20 to about 28, about 22 to about 27, about 28 to about 32, or about 31 kPa to about 33 kPa.

The pressure within the reactor can be reduced to less than atmospheric pressure using any suitable device, system, or combination of devices and/or systems. For example, the pressure can be reduced with one or more ejectors, e.g., a steam ejector, in fluid communication with the reactor and configured to pull a vacuum on the reactor. Prior to or upon introduction of the alkane to the reactor, the ejector or other vacuum device and/or system can be isolated from the reactor to reduce or avoid a loss of the dehydrogenated product. The dehydrogenated product can be recovered from the reactor and introduced to a compressor to produce a pressurized product.

When the amount of coke deposited on, in, or otherwise about the catalyst and/or the temperature of the catalyst reduces such that the reaction rate and/or conversion rate of the alkane to the alkene falls below a desired level, the alkane can be diverted to another reactor. The coked catalyst can be contacted with a purge fluid to remove at least a portion of any residual alkane on, about, and/or within the catalyst and/or within the reactor. The purge fluid can be or include, but is not limited to, nitrogen, argon, helium, carbon dioxide, exhaust or combustion gas, air, water or steam, or any combination thereof. In at least one example, the purge fluid can be or include steam. The purge fluid, e.g., steam, can be at a temperature of about 120° C., about 150° C., about 175° C., about 200° C., about 225° C., about 250° C., or about 275° C. to about 400° C., about 450° C., about 500° C., about 525° C., about 550° C., about 575° C., or about 600° C. For example, the purge fluid, e.g., steam, can be at a pressure of about 101 kPa, about 125 kPa, about 200 kPa, about 350 kPa, about 500 kPa, about 1,000 kPa, about 1,500 kPa, about 2,000 kPa, or about 2,500 kPa to about 3,000 kPa, about 3,500 kPa, about 4,000 kPa, or about 4,500 kPa.

After contacting the catalyst with the purge fluid, the catalyst can be decoked. Decoking the coked catalyst can remove at least a portion of the coke deposited on, in, and/or about the catalyst. The coke can be removed from the catalyst by combustion of the coke. For example, the catalyst can be contacted with an oxygen-containing gas or fluid at conditions sufficient to combust at least a portion of the coke, thereby removing coke from the coked catalyst. Illustrative oxygen-containing fluids can be or include, but are not limited to, ambient air, bottled air, in-line or in-house air, oxygen (O₂), nitrous oxide, ozone, or any mixture thereof. In at least one example, the oxygen-containing fluid can be or include air.

The oxygen-containing fluid can be at a temperature of about 500° C., about 550° C., about 600° C., about 650° C., about 660° C., about 665° C., about 670° C., or about 675° C. to a temperature of about 680° C., about 685° C., about 690° C., about 695° C., about 700° C., about 725° C., about 750° C., about 775° C., or about 800° C. at the time the oxygen-containing fluid is introduced into the reactor. The oxygen-containing fluid can be at a pressure of about 101 kPa, about 250 kPa, about 500 kPa, or about 750 kPa to about 1,000 kPa, about 1,250 kPa, about 1,500 kPa, about 1,750 kPa, about 2,500 kPa, about 3,000 kPa, about 3,500 kPa, about 4,000 kPa, or about 4,500 kPa at the time the oxygen-containing fluid is introduced into the reactor. For example, the oxygen-containing fluid can be at a pressure of about 115 kPa to about 200 kPa, about 117 kPa to about 1,702 kPa, about 117 kPa to about 170 kPa, about 400 kPa to about 1,200 kPa, about 800 kPa to about 1,600 kPa, or about 1,200 kPa to about 1,700 kPa at the time the oxygen-containing fluid is introduced into the reactor.

The oxygen-containing fluid can be at a temperature of about 500° C., about 550° C., about 600° C., about 650° C., about 660° C., about 665° C., about 670° C., or about 675° C. to a temperature of about 680° C., about 685° C., about 690° C., about 695° C., about 700° C., about 725° C., about 750° C., about 775° C., or about 800° C. when contacted with the coked catalyst to produce the regenerated catalyst. The oxygen-containing fluid can be at a pressure of about 101 kPa, about 250 kPa, about 500 kPa, or about 750 kPa to about 1,000 kPa, about 1,250 kPa, about 1,500 kPa, about 1,750 kPa, about 2,500 kPa, about 3,000 kPa, about 3,500 kPa, about 4,000 kPa, or about 4,500 kPa when contacted with the coked catalyst to produce the regenerated catalyst. For example, the oxygen-containing fluid can be at a pressure of about 115 kPa to about 200 kPa, about 117 kPa to about 1,702 kPa, about 117 kPa to about 170 kPa, about 400 kPa to about 1,200 kPa, about 800 kPa to about 1,600 kPa, or about 1,200 kPa to about 1,700 kPa when contacted with the coked catalyst to produce the regenerated catalyst.

The amount of the oxygen-containing fluid contacted with the coked catalyst to produce the decoked catalyst can widely vary. For example, the amount of the oxygen-containing fluid contacted with the coked catalyst to produce the decoked catalyst can be about 125 kg/s, about 175 kg/s, about 225 kg/s, about 250 kg/s, about 275 kg/s, about 300 kg/s, or about 325 kg/s to about 375 kg/s, about 400 kg/s, about 425 kg/s, about 450 kg/s, about 500 kg/s, about 550 kg/s, about 600 kg/s, or about 650 kg/s. In another example, the amount of the oxygen-containing fluid contacted with the coked catalyst to produce the decoked catalyst can be about 125 kg/s to about 220 kg/s, about 160 kg/s to about 260 kg/s, about 280 kg/s to about 420 kg/s, about 340 kg/s to about 360 kg/s, about 350 kg/s to about 370 kg/s, about 330 kg/s to about 355 kg/s, about 290 kg/s to about 320 kg/s, about 360 kg/s to about 410 kg/s, about 440 kg/s to about 540 kg/s, or about 520 kg/s to about 640 kg/s.

The length or period of time the oxygen-containing fluid can be contacted with the coked catalyst to produce the decoked catalyst can widely vary. For example, the oxygen-containing fluid can be contacted with the coked catalyst for a period of time of 400 seconds, about 450 seconds, about 475 seconds, about 500 seconds, or about 525 seconds to about 550 seconds, about 575 seconds, about 600 seconds, about 650 seconds, about 700 seconds, about 750 seconds, about 800 seconds, about 850 seconds, about 900 seconds, about 950 seconds, or about 1,000 seconds. In another example, the oxygen-containing fluid can be contacted with the coked catalyst for a period of time of about 420 seconds to about 520 seconds, about 460 seconds to about 560 seconds, about 490 seconds to about 590 seconds, about 520 seconds to about 620 seconds, about 520 seconds to about 560 seconds, about 580 seconds to about 620 seconds, about 610 seconds to about 660 seconds, about 640 seconds to about 800 seconds, about 710 seconds to about 900 seconds, or about 770 seconds to about 980 seconds to produce the decoked catalyst.

In one or more examples, one or more hydrocarbon fuels can be contacted with the coked catalyst while the oxygen-containing fluid contacts the coked catalyst. The hydrocarbon fuel can combust in the presence of the oxygen-containing fluid to increase the temperature of the decoked catalyst. In one or more examples, contact between the oxygen-containing fluid and the coked catalyst can be started, then contact between the hydrocarbon fuel and the coked catalyst can be started, then contact between the hydrocarbon fuel can be stopped, and then contact between the oxygen-containing fluid can be stopped. In another example, contact between the oxygen-containing fluid and the hydrocarbon fuel can be started at the same or substantially the same time, e.g., within 10 seconds of one another, then contact between the hydrocarbon fuel can be stopped, and then contact between the oxygen-containing fluid can be stopped.

The hydrocarbon fuel can be a gas and/or a liquid hydrocarbon. Illustrative hydrocarbon fuels can include, but are not limited to, one or more C₁-C₆ hydrocarbons such as methane, ethane, propane, butane, pentane, hexane, or any mixture thereof. In at least one example, the hydrocarbon fuel can be or include methane and can be contacted with the coked catalyst while the oxygen-containing fluid contacts the coked catalyst. In another example, the hydrocarbon fuel can include at least 90 wt %, at least 93 wt %, at least 95 wt %, at least 97 wt %, or at least 99 wt % methane. In at least one other example, the hydrocarbon fuel can be or include natural gas.

In one or more examples, the hydrocarbon fuel can be contacted with the coked catalyst for about 50%, about 55%, about 60%, about 65%, about 70%, about 75%, or about 78% to about 83%, about 85%, about 87%, about 90%, about 93%, about 95%, about 97%, about 99%, or more of the period of time the oxygen-containing fluid contacts the coked catalyst. In another example, the hydrocarbon fuel can be contacted with the coked catalyst for about 50% to about 76%, about 50% to about 77%, about 65% to about 85%, about 75% to about 80%, about 77% to about 92%, about 82% to about 96%, about 78% to about 84%, about 83% to about 91%, about 86% to about 93%, about 88% to about 96%, about 89% to about 97%, about 92% to about 99%, about 94% to about 100%, about 96% to about 99.5%, about 98% to about 99%, or about 97% to about 99% of the period of time the oxygen-containing fluid contacts the coked catalyst. In other examples, the hydrocarbon fuel can be contacted with the coked catalyst for at least 77%, at least 80%, at least 83%, at least 85%, at least 87%, at least 90%, at least 93%, at least 95%, at least 97%, or at least 99% of the period of time the oxygen-containing fluid contacts the coked catalyst.

In one or more examples, contacting the coked catalyst with the hydrocarbon fuel for greater than 77% of the period of time the oxygen-containing fluid contacts the coked catalyst can increase the amount of the one or more alkenes in the dehydrogenated product as compared to introducing the hydrocarbon fuel for only 77% of the period of time the oxygen-containing fluid contacts the coked catalyst. For example, contacting the coked catalyst with the hydrocarbon fuel for greater than 77% of the period of time the oxygen-containing fluid contacts the coked catalyst can increase the amount of the one or more alkenes in the dehydrogenated product by at least 0.5 wt %, at least 1 wt %, at least 1.5 wt %, at least 2 wt %, at least 2.5 wt %, or at least 3 wt % to about 5 wt %, about 7 wt %, about 9 wt %, about 11 wt %, about 13 wt %, about 15 wt %, or about 17 wt % as compared to contacting the coked catalyst with the hydrocarbon fuel for only 77% of the period of time the oxygen-containing fluid contacts the coked catalyst. In one or more examples, contacting the coked catalyst with the hydrocarbon fuel for greater than 80% of the period of time the oxygen-containing fluid contacts the coked catalyst can increase the amount of the one or more alkenes in the dehydrogenated product as compared to introducing the hydrocarbon fuel for only 77% of the period of time the oxygen-containing fluid contacts the coked catalyst. For example, contacting the coked catalyst with the hydrocarbon fuel for greater than 80% of the period of time the oxygen-containing fluid contacts the coked catalyst can increase the amount of the one or more alkenes in the dehydrogenated product by at least 0.5 wt %, at least 1 wt %, at least 1.5 wt %, at least 2 wt %, at least 2.5 wt %, or at least 3 wt % to about 5 wt %, about 7 wt %, about 9 wt %, about 11 wt %, about 13 wt %, about 15 wt %, or about 17 wt % as compared to contacting the coked catalyst with the hydrocarbon fuel for only 77% of the period of time the oxygen-containing fluid contacts the coked catalyst. In one or more examples, contacting the coked catalyst with the hydrocarbon fuel for greater than 85% of the period of time the oxygen-containing fluid contacts the coked catalyst can increase the amount of the one or more alkenes in the dehydrogenated product as compared to introducing the hydrocarbon fuel for only 77% of the period of time the oxygen-containing fluid contacts the coked catalyst. For example, contacting the coked catalyst with the hydrocarbon fuel for greater than 85% of the period of time the oxygen-containing fluid contacts the coked catalyst can increase the amount of the one or more alkenes in the dehydrogenated product by at least 0.5 wt %, at least 1 wt %, at least 1.5 wt %, at least 2 wt %, at least 2.5 wt %, or at least 3 wt % to about 5 wt %, about 7 wt %, about 9 wt %, about 11 wt %, about 13 wt %, about 15 wt %, or about 17 wt % as compared to contacting the coked catalyst with the hydrocarbon fuel for only 77% of the period of time the oxygen-containing fluid contacts the coked catalyst. In one or more examples, contacting the coked catalyst with the hydrocarbon fuel for greater than 90% of the period of time the oxygen-containing fluid contacts the coked catalyst can increase the amount of the one or more alkenes in the dehydrogenated product as compared to introducing the hydrocarbon fuel for only 77% of the period of time the oxygen-containing fluid contacts the coked catalyst. For example, contacting the coked catalyst with the hydrocarbon fuel for greater than 90% of the period of time the oxygen-containing fluid contacts the coked catalyst can increase the amount of the one or more alkenes in the dehydrogenated product by at least 0.5 wt %, at least 1 wt %, at least 1.5 wt %, at least 2 wt %, at least 2.5 wt %, or at least 3 wt % to about 5 wt %, about 7 wt %, about 9 wt %, about 11 wt %, about 13 wt %, about 15 wt %, or about 17 wt % as compared to contacting the coked catalyst with the hydrocarbon fuel for only 77% of the period of time the oxygen-containing fluid contacts the coked catalyst. In one or more examples, contacting the coked catalyst with the hydrocarbon fuel for greater than 95% of the period of time the oxygen-containing fluid contacts the coked catalyst can increase the amount of the one or more alkenes in the dehydrogenated product as compared to introducing the hydrocarbon fuel for only 77% of the period of time the oxygen-containing fluid contacts the coked catalyst. For example, contacting the coked catalyst with the hydrocarbon fuel for greater than 95% of the period of time the oxygen-containing fluid contacts the coked catalyst can increase the amount of the one or more alkenes in the dehydrogenated product by at least 0.5 wt %, at least 1 wt %, at least 1.5 wt %, at least 2 wt %, at least 2.5 wt %, or at least 3 wt % to about 5 wt %, about 7 wt %, about 9 wt %, about 11 wt %, about 13 wt %, about 15 wt %, or about 17 wt % as compared to contacting the coked catalyst with the hydrocarbon fuel for only 77% of the period of time the oxygen-containing fluid contacts the coked catalyst. In one or more examples, contacting the coked catalyst with the hydrocarbon fuel for greater than 98% of the period of time the oxygen-containing fluid contacts the coked catalyst can increase the amount of the one or more alkenes in the dehydrogenated product as compared to introducing the hydrocarbon fuel for only 77% of the period of time the oxygen-containing fluid contacts the coked catalyst. For example, contacting the coked catalyst with the hydrocarbon fuel for greater than 98% of the period of time the oxygen-containing fluid contacts the coked catalyst can increase the amount of the one or more alkenes in the dehydrogenated product by at least 0.5 wt %, at least 1 wt %, at least 1.5 wt %, at least 2 wt %, at least 2.5 wt %, or at least 3 wt % to about 5 wt %, about 7 wt %, about 9 wt %, about 11 wt %, about 13 wt %, about 15 wt %, or about 17 wt % as compared to contacting the coked catalyst with the hydrocarbon fuel for only 77% of the period of time the oxygen-containing fluid contacts the coked catalyst.

In one or more examples, contacting the coked catalyst with the hydrocarbon fuel for at least 80% of the period of time the oxygen-containing fluid contacts the coked catalyst can increase the amount of the one or more alkenes in the dehydrogenated product as compared to not introducing the hydrocarbon fuel or to introducing the hydrocarbon fuel for only 77% or less of the period of time the oxygen-containing fluid contacts the coked catalyst. For example, contacting the coked catalyst with the hydrocarbon fuel for at least 85% of the period of time the oxygen-containing fluid contacts the coked catalyst can increase the amount of the one or more alkenes in the dehydrogenated product by at least 0.5 wt %, at least 1 wt %, at least 1.5 wt %, at least 2 wt %, at least 2.5 wt %, or at least 3 wt % to about 5 wt %, about 7 wt %, about 9 wt %, about 11 wt %, about 13 wt %, about 15 wt %, or about 17 wt % as compared to contacting the coked catalyst with the hydrocarbon fuel for only 77% or less of the period of time the oxygen-containing fluid contacts the coked catalyst. In another example, contacting the coked catalyst with the hydrocarbon fuel for at least 90% of the period of time the oxygen-containing fluid contacts the coked catalyst can increase the amount of the one or more alkenes in the dehydrogenated product by at least 0.5 wt %, at least 1 wt %, at least 1.5 wt %, at least 2 wt %, at least 2.5 wt %, or at least 3 wt % to about 5 wt %, about 7 wt %, about 9 wt %, about 11 wt %, about 13 wt %, about 15 wt %, or about 17 wt % as compared to contacting the coked catalyst with the hydrocarbon fuel for only 77% or less of the period of time the oxygen-containing fluid contacts the coked catalyst. In another example, contacting the coked catalyst with the hydrocarbon fuel for at least 95% of the period of time the oxygen-containing fluid contacts the coked catalyst can increase the amount of the one or more alkenes in the dehydrogenated product by at least 0.5 wt %, at least 1 wt %, at least 1.5 wt %, at least 2 wt %, at least 2.5 wt %, or at least 3 wt % to about 5 wt %, about 7 wt %, about 9 wt %, about 11 wt %, about 13 wt %, about 15 wt %, or about 17 wt % as compared to contacting the coked catalyst with the hydrocarbon fuel for only 77% or less of the period of time the oxygen-containing fluid contacts the coked catalyst. In another example, contacting the coked catalyst with the hydrocarbon fuel for at least 98% of the period of time the oxygen-containing fluid contacts the coked catalyst can increase the amount of the one or more alkenes in the dehydrogenated product by at least 0.5 wt %, at least 1 wt %, at least 1.5 wt %, at least 2 wt %, at least 2.5 wt %, or at least 3 wt % to about 5 wt %, about 7 wt %, about 9 wt %, about 11 wt %, about 13 wt %, about 15 wt %, or about 17 wt % as compared to contacting the coked catalyst with the hydrocarbon fuel for only 77% or less of the period of time the oxygen-containing fluid contacts the coked catalyst. In another example, contacting the coked catalyst with the hydrocarbon fuel for at least 85%, at least 87%, at least 89%, at least 91%, at least 93%, at least 95%, at least 97%, or at least 99% of the period of time the oxygen-containing fluid contacts the coked catalyst can increase the amount of the one or more alkenes in the dehydrogenated product by at least 0.5 wt %, at least 1 wt %, at least 1.5 wt %, at least 2 wt %, at least 2.5 wt %, or at least 3 wt % to about 5 wt %, about 7 wt %, about 9 wt %, about 11 wt %, about 13 wt %, about 15 wt %, or about 17 wt % as compared to contacting the coked catalyst with the hydrocarbon fuel for only 77% or less of the period of time the oxygen-containing fluid contacts the coked catalyst.

In another example, contacting the coked catalyst with the hydrocarbon fuel for at least 90% of the period of time the oxygen-containing fluid contacts the coked catalyst can increase the amount of the one or more alkenes in the dehydrogenated product by at least 0.5 wt %, at least 1 wt %, at least 1.5 wt %, at least 2 wt %, at least 2.5 wt %, or at least 3 wt % to about 5 wt %, about 7 wt %, about 9 wt %, about 11 wt %, about 13 wt %, about 15 wt %, or about 17 wt % as compared to contacting the coked catalyst with the hydrocarbon fuel for only 77% or less of the period of time the oxygen-containing fluid contacts the coked catalyst. In another example, contacting the coked catalyst with the hydrocarbon fuel for at least 95% of the period of time the oxygen-containing fluid contacts the coked catalyst can increase the amount of the one or more alkenes in the dehydrogenated product by at least 0.5 wt %, at least 1 wt %, at least 1.5 wt %, at least 2 wt %, at least 2.5 wt %, or at least 3 wt % to about 5 wt %, about 7 wt %, about 9 wt %, about 11 wt %, about 13 wt %, about 15 wt %, or about 17 wt % as compared to contacting the coked catalyst with the hydrocarbon fuel for only 77% or less of the period of time the oxygen-containing fluid contacts the coked catalyst. In another example, contacting the coked catalyst with the hydrocarbon fuel for at least 98% of the period of time the oxygen-containing fluid contacts the coked catalyst can increase the amount of the one or more alkenes in the dehydrogenated product by at least 0.5 wt %, at least 1 wt %, at least 1.5 wt %, at least 2 wt %, at least 2.5 wt %, or at least 3 wt % to about 5 wt %, about 7 wt %, about 9 wt %, about 11 wt %, about 13 wt %, about 15 wt %, or about 17 wt % as compared to contacting the coked catalyst with the hydrocarbon fuel for only 77% or less of the period of time the oxygen-containing fluid contacts the coked catalyst. In another example, contacting the coked catalyst with the hydrocarbon fuel for at least 85%, at least 87%, at least 89%, at least 91%, at least 93%, at least 95%, at least 97%, or at least 99% of the period of time the oxygen-containing fluid contacts the coked catalyst can increase the amount of the one or more alkenes in the dehydrogenated product by at least 0.5 wt %, at least 1 wt %, at least 1.5 wt %, at least 2 wt %, at least 2.5 wt %, or at least 3 wt % to about 5 wt %, about 7 wt %, about 9 wt %, about 11 wt %, about 13 wt %, about 15 wt %, or about 17 wt % as compared to contacting the coked catalyst with the hydrocarbon fuel for only 77% or less of the period of time the oxygen-containing fluid contacts the coked catalyst.

The decoked catalyst can be contacted with one or more reducing gases to produce a regenerated catalyst and an off-gas. For example, the reducing gas can be introduced into the reactor and can contact the decoked catalyst therein to produce the regenerated catalyst and the off-gas. The reducing gas can remove at least a portion of any adsorbed oxygen from the decoked catalyst. For example, if the catalyst includes chromium, the reducing gas can convert at least a portion of any chromium (VI) to chromium (III). The off-gas can include at least a portion of the adsorbed oxygen that can be removed from the decoked catalyst.

Illustrative reducing gases can be or include, but are not limited to, hydrogen, methane, ethane, ethylene, propane, propylene, carbon monoxide, butane, or any mixture thereof. For example, the reducing gas can be or include a hydrogen product recovered from one or more pressure swing absorption units that separates a gas mixture containing hydrogen and one or more other components such as methane, ethane, carbon dioxide, nitrogen, oxygen, ammonia, or any mixture thereof. In another example, the reducing gas can be or include hydrogen recovered via membrane separation of a gas mixture containing hydrogen and one or more other components such as methane, ethane, carbon dioxide, nitrogen, oxygen, ammonia, or any mixture thereof. In one or more embodiments, the reducing gas can include at least 70 wt % of hydrogen, at least 73 wt % of hydrogen, at least 75 wt % of hydrogen, at least 77 wt % of hydrogen, at least 80 wt % of hydrogen, at least 83 wt % of hydrogen, at least 85 wt % of hydrogen, at least 87 wt % of hydrogen, at least 90 wt % of hydrogen, at least 91 wt % of hydrogen, at least 92 wt % of hydrogen, at least 93 wt % of hydrogen, at least 94 wt % of hydrogen, at least 95 wt % of hydrogen, at least 96 wt % of hydrogen, at least 97 wt % of hydrogen, at least 98 wt % of hydrogen, at least 98.5 wt % of hydrogen, at least 99 wt % of hydrogen, at least 99.5 wt % of hydrogen, at least 99.9 wt % of hydrogen, or at least 99.99 wt % of hydrogen. In another example, the reducing gas can be or include a tail gas that includes about 40 wt % to about 60 wt % of hydrogen. In another example, the reducing gas can be or include a gas mixture, e.g., a tail gas, an off-gas, a process gas, or any mixture thereof, that includes about 40 wt % of hydrogen or more, about 45 wt % of hydrogen or more, about 50 wt % of hydrogen or more, about 55 wt % of hydrogen or more, about 60 wt % of hydrogen or more, about 65 wt % of hydrogen or more, about 70 wt % of hydrogen or more, or about 75 wt % of hydrogen or more.

The introduction of the reducing gas can be stopped at least 10 seconds, at least 12 seconds, at least 14 seconds, at least 16 seconds, at least 18 seconds, at least 20 seconds, at least 22 seconds, at least 24 seconds, at least 26 seconds, at least 28 seconds, at least 30 seconds, at least 32 seconds, at least 34 seconds, at least 36 seconds, at least 38 seconds, at least 40 seconds, at least 42 seconds, at least 44 seconds, at least 46 seconds, at least 48 seconds, at least 50 seconds, at least 52 seconds, at least 54 seconds, at least 56 seconds, at least 58 seconds, or at least 60 seconds, prior to or before the alkane can be re-introduced into the reactor. For example, the introduction of the reducing gas can be stopped at least 10 seconds, about 11 seconds, about 13 seconds, about 14 seconds, about 15 seconds, or about 18 seconds to about 20 seconds, about 26 seconds, about 30 seconds, about 35 seconds, about 40 seconds, about 45 seconds, about 50 seconds, about 55 seconds, or about 60 seconds prior to or before the alkane is re-introduced into the reactor. In another example, the introduction of the reducing gas can be stopped at least 28 seconds, about 30 seconds, about 35 seconds, about 40 seconds, about 45 seconds, or about 50 seconds to about 55 seconds, about 60 seconds, about 65 seconds, about 70 seconds, or about 75 seconds prior to or before the alkane is re-introduced into the reactor. In another example, the introduction of the reducing gas can be stopped at least 10 seconds, at least 12 seconds, at least 14 seconds, at least 16 seconds, at least 18 seconds, at least 20 seconds, at least 22 seconds, at least 24 seconds, at least 26 seconds, at least 28 seconds, or at least 30 seconds to about 35 seconds, about 40 seconds, about 45 seconds, about 50 seconds, about 55 seconds, about 60 seconds, about 65 seconds, about 70 seconds, or about 75 seconds prior to or before the alkane is re-introduced into the reactor.

In one or more embodiments, an off-gas from the reactor can be recovered while the reducing gas is introduced into the reactor. The off-gas can be recovered by exposing the reactor to a vacuum or evacuation source, e.g., an ejector. The off-gas recovered from the reactor while the reducing gas is introduced into the reactor can be vented directly to the atmosphere. For example, the off-gas recovered from the reactor, while the reducing gas is introduced into the reactor, can be vented directly to the atmosphere without passing through a waste heat boiler. In another example the off-gas can be recovered from the reactor or other process environment when the reducing gas is introduced into the reactor or other process environment and the off-gas can be introduced directly to the atmosphere without passing through a waste heat boiler or any other apparatus or system. The off-gas, when introduced directly to the atmosphere, can have the same or substantially the same composition as the off-gas has when in the reactor or other process environment.

When the off-gas recovered from the reactor (while the reducing gas is introduced to the reactor) is introduced directly to the atmosphere without passing through a waste heat boiler, the reducing gas can include at least 70 wt % of hydrogen, at least 80 wt % of hydrogen, at least 90 wt % of hydrogen, at least 95 wt % of hydrogen, at least 97 wt % of hydrogen, at least 99 wt % of hydrogen, at least 99.9 wt % of hydrogen, or at least 99.99 wt % of hydrogen. Introduction of the reducing gas to the reactor can be stopped, but recovery of the off-gas from the reactor can be continued for a period of time by maintaining exposure of the reactor to the vacuum or evacuation source. For example, after introduction of the reducing gas to the reactor stops, the off-gas can continue to be recovered from the reactor by maintaining exposure of the reactor to the vacuum or evacuation source for a period time of at least 5 seconds, at least 10 seconds, at least 15 seconds, at least 20 seconds, at least 25 seconds, at least 30 seconds, at least 35 seconds, at least 40 seconds, or at least 45 seconds. After the period of time the reactor is exposed to the vacuum or evacuation source ends, additional alkane can be introduced to the reactor or other process environment and contacted with the regenerated catalyst to produce additional dehydrogenated product and re-coked catalyst. The re-coked catalyst can be processed again to produce regenerated catalyst again.

The FIGURE depicts a schematic of an illustrative system 100 for dehydrogenating one or more alkanes (e.g., propane and/or butane) to produce a dehydrogenated product that can include one or more alkenes (e.g., propylene and/or butylene), according to one or more embodiments. One or more alkanes can be stored or otherwise contained in an alkane source 102 fluidly coupled via line 104 to a heating side of a recuperator or heat exchanger 106, a heater 107, a valve 108, and a reactor 110. The alkanes can be transferred via line 104 from the alkane source 102 through the heating side of the heat exchanger 106 and the heater 107. The alkanes can be heated when passing through the heating side of the heat exchanger 106 and the heater 107. The alkanes can also be transferred via line 104 from the heater 107 and into the reactor 110. The valve 108 can be operably coupled to the line 104 and disposed between the heater 107 and the reactor 110. The valve 108 can be used to control the flow rate of the alkanes entering the reactor 110 including starting and stopping the introduction of the alkanes entering the reactor 110.

The reactor 110 can include one or more catalysts in one or more catalyst beds 112. The alkanes can pass through the catalyst bed 112 within the reactor 110 and can be exposed to or otherwise make contact with the catalyst in the catalyst bed 112. In one example, the alkanes can be introduced into the reactor 110 having regenerated catalyst disposed on or in the catalyst bed 112 which can produce a dehydrogenated product that can have one or more alkenes, such as propylene or butylene.

The dehydrogenated product having one or more alkenes, as well as any unreacted alkanes and/or other precursors or products, can be removed or otherwise transferred from the reactor 110 as a crude product stream. This crude product stream via line 113 can be transferred from the reactor 110, through a cooling side of the heat exchanger 106, a compressor 115, and into a separator 116. The crude product stream can be thermally cooled when passing through the cooling side of the heat exchanger 106 and the heater 107. Energy can be indirectly transferred from the crude product stream in line 113 passing through the cooling side of the heat exchanger 106 to the alkane feed in the line 104 passing through the heating side of the heat exchanger 106. A valve 114 can be operably coupled to the line 113 and disposed between the reactor 110 and the cooling side of the heat exchanger 106. The valve 114 can be used to control the flow rate of the dehydrogenated products or precursors evacuating or otherwise exiting the reactor 110.

The crude product stream can be compressed by the compressor 115 and transferred into the separator 116 where the crude product stream can be separated into two or more components. In one example, the crude product stream can be separated into a first stream containing the purified dehydrogenated products having one or more alkenes (e.g., propylene and/or butylene) that can be transferred from the separator 116 to a product tank 118 and a second stream containing one or more alkanes (e.g., propane and/or butane) or other precursors, that can be transferred from the separator 116 to a line 117. As depicted in the FIGURE, line 117 can be fluidly coupled to and between the separator 116 and line 104, such that unreacted and separated alkanes can flow into the alkane stream passing between the alkane source 102 and the heating side of the heat exchanger 106.

In one or more examples, the system 100 can include a hydrocarbon fuel source 130 fluidly coupled via line 132 to the reactor 110. The hydrocarbon fuel source 130 can contain one or more hydrocarbon fuels, such as, but not limited to, natural gas, methane, ethane, acetylene, or any combination thereof. The hydrocarbon fuels can be transferred via line 132 from the hydrocarbon fuel source 130 and into the reactor 110. A valve 138 can be operably coupled to the line 132 and disposed between the hydrocarbon fuel source 130 and the reactor 110. The valve 138 can be used to control the flow rate of the hydrocarbon fuels entering the reactor 110 including starting and stopping the administration of the hydrocarbon fuels entering the reactor 110.

In other examples, the system 100 can also include an oxygen-containing fluid source 140, a compressor 144, and a heater 146 fluidly coupled to the reactor 110 via line 142. The oxygen-containing fluid source 140 can be or include one or more air or oxygen-containing gases or fluids, such as, but not limited to, ambient air, bottled air, in-line or in-house air, oxygen (O₂), nitrous oxide, ozone, or any mixture thereof. The oxygen-containing fluid can be transferred from the oxygen-containing fluid source 140, through the compressor 144 and the heater 146, and into the reactor 110 via line 142. The compressor 144 can pressurize the oxygen-containing fluid and the heater 146 can increase the temperature of the oxygen-containing fluid by adding thermal energy thereto—both upstream of the reactor 110. The incoming oxygen-containing fluid, therefore, can be pressurized and heated to the respective pressure and temperature desired in the reactor 110. A valve 148 can be operably coupled to the line 142 and disposed between the oxygen-containing fluid source 140 and the reactor 110. The valve 148 can be used to control the flow rate of the oxygen-containing fluid entering the reactor 110 including starting and stopping the administration of the oxygen-containing fluid entering the reactor 110.

In one or more examples, the system 100 can also include a purge fluid source 150 fluidly coupled to the reactor 110 via line 152. The purge fluid source 150 (e.g., sparger purge source) can contain one or more sources of purge gas or fluids, such as, but not limited to, nitrogen, argon, helium, carbon dioxide, exhaust or combustion gas, air, water or steam, or any combination thereof. In some examples, the purge fluid source 150 can include nitrogen or argon gas. The purge fluid can be transferred via line 152 from the purge fluid source 150 and into the reactor 110. A valve 158 can be operably coupled to the line 152 and disposed between the purge fluid source 150 and the reactor 110. The valve 158 can be used to control the flow rate of the purge fluid entering the reactor 110 including starting and stopping the administration of the purge fluid entering the reactor 110. In some examples, the reactor 110 can be exposed to a sparger purge of nitrogen gas administered by the valve 158.

In other embodiments, the system 100 can also include a reducing gas source 160 fluidly coupled via line 162 to the reactor 110. The reducing gas source 160 can contain one or more sources of a reducing gas, liquid, or fluid that can include one or more reducing agents, such as, but not limited to, hydrogen (H₂). The reducing gas can be transferred via line 162 from the reducing gas source 160 and into the reactor 110. A valve 168 can be operably coupled to the line 162 and disposed between the reducing gas source 160 and the reactor 110. The valve 168 can be used to control the flow rate of the reducing gas entering the reactor 110 including starting and stopping the administration of the reducing gas entering the reactor 110.

In one or more examples, the system 100 can also include a steam source 170 fluidly coupled via line 172 to the reactor 110. The steam source 170 can contain one or more sources of steam or water (e.g., liquid, gas, or fluid states), such as, but not limited to, steam, water, or a mixture thereof. In some examples, the steam source 170 can include pressurized and/or heated steam, such as, but not limited to, steam generator, in-line or in-house, feed from another system, or any combination thereof. The steam can be transferred from the steam source 170 and into the reactor 110 via line 172. A valve 178 can be operably coupled to the line 172 and disposed between the steam source 170 and the reactor 110. The valve 178 can be used to control the flow rate of the steam entering the reactor 110 including starting and stopping the administration of the steam entering the reactor 110.

In one or more examples, the hydrocarbon fuel source 130, the oxygen-containing fluid source 140, the purge fluid source 150, the reducing gas source 160, the steam source 170, or any combination thereof, can be used to administer one or more reagents or compounds into the reactor 110 and in contact with the catalyst beds 112 in order to chemically oxidize, chemically reduce, remove coke, regenerate, or otherwise process the catalysts disposed on or in the catalyst beds 112. An off-gas stream can be generated in the reactor 110 and can include, but is not limited to, gases, liquids, fluids, and/or solids of materials introduced or generated during any of these process steps, including, for example, during the decoking and regeneration processes. The off-gas stream can be removed via lines 123 and/or 125 from the reactor 110. A valve 124 can be operably coupled to the line 123 and disposed between the reactor 110 and an ejector 122. A valve 126 can also be operably coupled to the line 125 and disposed between the reactor 110 and a waste heat boiler 129. Each of the valves 124, 126 can independently be used to control the flow rate of the off-gases and/or undesirable materials evacuating or otherwise exiting the reactor 110 via lines 123, 125, respectively.

In one example, the off-gases can be transferred via line 123 to the ejector 122 prior to being directly emitted into the ambient atmosphere. A motive fluid source 121 can be used to drive or otherwise provide thrust or energy to the ejector 122. A valve 128 can be operably coupled to a line 127 and disposed between the ejector 122 and the ambient atmosphere. The valve 128 can be used to control the flow of the off-gas entering the ambient atmosphere via line 127. In another example, the off-gases can be transferred to the waste heat boiler 129 via line 125 where the off-gases can be treated to remove, dilute, or otherwise treat particulates, pollutants, or other compounds. Thereafter, a purified off-gas can be emitted from the waste heat boiler 129 into the ambient atmosphere via line 131.

In one or more examples, a control system 120 can be operatively connected with the system 100 to monitor, control, and/or adjust the components of the system 100 and operating parameters, including but not limited to, temperatures, pressures (including port, line, and device internal pressures), timing, valve control (including opening, closing, or modulating), flow meters and/or flowrates, compressor or pump operation, and/or gas or fluid administrations at or during start-up operations, shut-down operations, process operations, and/or anytime therebetween. The control system 120 may include one or more computer systems 119 with a multi-controller algorithm configured to monitor, actuate, adjust, manipulate, and/or otherwise control one or more components, parts, or portions of the system 100. The control system 120 may also be configured to implement one or more steps, methods, or processes for the system 100 including, but not limited to dehydrogenation, decoking, regeneration, purging, evacuating, and/or other steps, methods, or processes described herein.

The control system 120 can be communicably coupled through any suitable means including but not limited to, wired connections and/or wireless connections. The control system 120 can independently be communicably coupled to the temperature control devices, such as, but not limited to, the heat exchanger 106, the heater 107, and the heater 146, one or more pumps and/or compressors, such as, but not limited to, the compressors 115, 144, and other components, such as, but not limited to, the separator 116, the motive fluid source 121, the ejector 122, and/or the waste heat boiler 129. The control system 120 can also be communicably coupled to the valves 108, 114, 124, 126, 138, 148, 158, 168, and 178, such that each valve can independently be opened, closed, adjusted, modulated, or otherwise controlled by the control system 120. The control system 120 can also be communicably coupled to the alkane source 102, the hydrocarbon fuel source 130, the oxygen-containing fluid source 140, the purge fluid source 150, the reducing gas source 160, and the steam source 170.

In one or more examples, the control system 120 can be configured to actuate, adjust, manipulate, and/or otherwise control one or more parts and/or systems of the system 100. The control system 120 may also be configured to monitor one or more parameters and/or variables of the components within the system 100 including, but not limited to pressures, temperatures, and/or flowrates. By controlling the various part and/or systems of the system 100, the control system 120 may control the flow of the fluids and/or gases throughout the system 100, thereby regulating the temperature, pressures, and/or the physical state of the fluids and/or gases throughout the system 100.

In one or more examples, dehydrogenating one or more alkanes can include introducing the alkane via line 104 to the reactor 110. The alkane can be contacted with the catalyst in the catalyst bed 112 located within the reactor 110 to produce the dehydrogenated product that can be recovered via line 113. Dehydrogenation of the alkane can also deposit coke on the catalyst in the catalyst bed 112 to produce the coked catalyst. During introduction of the alkane via line 104 and recovery of the dehydrogenated product via line 113 valves 108 and 114 can be in the open state and valves 124, 126, 138, 148, 158, 168, and 178 can be in the closed state. The pressure within the reactor 110 can be under a vacuum, e.g., the pressure can be less than 38 kPa, less than 36 kPa, less than 33 kPa, less than 30 kPa, less than 28 kPa, or less than 25 kPa, at the time the alkane via line 104 is initially introduced to the reactor 110. The valves 108 and 114 can be closed and valves 158, 124, and 126 can be opened. Opening valve 158 can introduce the purge fluid via line 152 into the reactor 110 and/or opening valve 178 can introduce steam via line 172 into the reactor 110. Opening valve 124 can provide a vacuum or exhaustion via line 123 from the reactor 110 to the ejector 122 and opening valve 126 can provide a vacuum or exhaustion via line 125 from the reactor 110 to the waste heat boiler 129. The purge fluid and/or steam can flow through the reactor 110 and contact the coked catalyst to remove at least a portion of any residual alkane or alkene. The purge fluid via line 152 can be or include nitrogen gas and the steam via line 172 can be or include steam and/or water. The valves 158, 178 can be closed and the valve 148 can be opened to introduce the oxygen-containing fluid via line 142 to the reactor 110. The oxygen-containing fluid introduced via line 142 to the reactor 110 can be heated to a temperature of at least 600° C., e.g., via heat from compression in the compressor 144 and/or the heater 146. The oxygen-containing fluid can contact the coked catalyst to produce a decoked catalyst. For example, the coke disposed on, in, or otherwise about the coked catalyst can be combusted in the presence of the oxygen-containing fluid. Optionally, the hydrocarbon fuel via line 132 can be introduced to the reactor 110 by opening valve 138. The hydrocarbon fuel via line 132 introduced to the reactor 110 can be contacted with the coked catalyst 112 for at least 77%, at least 80%, at least 85%, at least 90%, at least 95%, at least 97%, at least 98%, at least 98.5%, or at least 99% of the time that the oxygen-containing fluid introduced via line 142 is contacted with the coked catalyst. The valve 148 and, if open, valve 138 can be closed. Valve 168 can be opened to introduce the reducing gas via line 162 to the reactor 110. The reducing gas via line 162 can contact the decoked catalyst to produce a regenerated catalyst. The reducing gas in line 162 can include at least 40 wt % of hydrogen, for example, but not limited to, about 60 wt % of hydrogen, about 70 wt % of hydrogen, about 80 wt % of hydrogen, about 85 wt % of hydrogen, about 90 wt % of hydrogen, about 93 wt % of hydrogen, about 98 wt % of hydrogen, about 99 wt % of hydrogen, about 99.9 wt % of hydrogen, or about 99.99 wt % of hydrogen. In another embodiment, the reducing gas in line 162 can include at least 90 wt % of hydrogen, for example, but not limited to, about 95 wt % of hydrogen, about 97 wt % of hydrogen, about 99 wt % of hydrogen, or about 99.9 wt % of hydrogen. Introduction of the reducing gas via line 162 to the reactor can be stopped by closing valve 168. Introduction of the reducing gas via line 162 to the reactor 110 can be stopped at least 10 seconds, at least 15 seconds, at least 20 seconds, at least 25 seconds, at least 28 seconds, at least 30 seconds, at least 35 seconds, at least 40 seconds, at least 45 seconds, or at least 50 seconds prior to re-introducing the alkane via line 104 to the reactor. Accordingly, valves 108, 114, 138, 148, 158, and 178 can be closed and valves 124 and/or 126 can be open for at least 10 seconds prior to re-introducing the alkane to the reactor 110. In another example, valves 108, 114, 138, 148, 158, 178, 124 and 126 can all be closed for at least a portion of the time, e.g., at least 10 seconds at least 15 seconds, at least 18 seconds, at least 20 seconds, or at least 25 seconds, between when the reducing gas via line 162 is stopped and the alkane is reintroduced via line 104 to the reactor 110. During introduction of the reducing gas via line 162 to the reactor 110, the off-gas recovered via line 123 from the reactor can flow through the ejector 122 and can be introduced directly to the atmosphere via line 127. For example, during introduction of the reducing gas via line 162 to the reactor 110 the off-gas recovered via line 123 from the reactor can flow through the ejector 122 and be introduced directly to the atmosphere via line 127 without passing through the waste heat boiler 129. In some examples, the off-gas can be introduced to the atmosphere without ever contacting a catalyst prior to being introduced into the atmosphere. In another example, during the introduction of the reducing gas via line 162 to the reactor 110 the off-gas recovered via line 125 from the reactor 110 can flow through the waste heat boiler 129 and the purified or otherwise processed off-gas can be introduced to the atmosphere via line 131.

In one or more examples, the system 100 can include one or more of any of the components depicted or discussed herein. In one embodiment, the system 100 can include two or more reactors 110, where each of the reactors can include one or more the catalyst beds 112. In some examples, the system 100 can include 2, 3, 4, 5, 6, 7, 8, 9, 10, 11, 12, 13, 14, 15, 16, 17, 18, 19, 20, or more reactors 110. For example, the system 100 can include 2 to about 50, 2 to about 40, 2 to about 30, 2 to about 20, 2 to 12, 2 to 11, 2 to 10, 2 to 9, 2 to 8, 2 to 7.2 to 6.2 to 5.2 to 4.2 to 3.4 to 12.4 to 11.4 to 10.4 to 9.4 to 8.4 to 7.4 to 6.4 to 5, 6 to 12, 6 to 11, 6 to 10, 6 to 9, or 6 to 8.

In one or more examples, the reactor 110 can be operated such that a continuous process can be carried out. For example, introduction of the alkane via line 104 to the reactor 110, contacting the alkane with the regenerated catalyst in the catalyst bed 112, contacting the coked catalyst in the catalyst bed 112 with the purge fluid, e.g., steam via line 172 and/or nitrogen via line 152, contacting the coked catalyst in the catalyst bed 112 with the oxygen-containing fluid via line 142 and, optionally the hydrocarbon fuel via line 132, introduction of the reducing gas line 162 to the reactor 110, recovery of the off-gas via line 131 from the reactor 110, and venting the recovered off-gas via line 131 directly to the atmosphere can be repeated a plurality of times, e.g., at least 50 times, at least 100 times, at least 150 times, at least 200 times, at least 250 times, at least 300 times, at least 400 times, at least 500 times, at least 1,000 times, at least 2,000 times or more. The steps can be repeated a plurality of times to provide a continuous process. The steps can be sequentially repeated a plurality of times to provide a continuous process. In another example, the alkane via line 104 can be introduced to the reactor 110, the alkane can be contacted with the regenerated catalyst in the catalyst bed 112, the coked catalyst in the catalyst bed 112 can be contacted with the purge fluid, e.g., steam via line 172 and/or nitrogen via line 152, the coked catalyst in the catalyst bed 112 can be contacted with the oxygen-containing fluid via line 142, the reducing gas can be introduced via line 162 to the reactor 110, the off-gas via line 131 from the reactor 110 can be recovered and vented directly to the atmosphere can be repeated at least 20 times, at least 25 times, at least 30 times, at least 35 times, at least 40 times, at least 45 times, 50 times, at least 55 times, at least 60 times, at least 70 times, at least 80 times, at least 100 times, at least 125 times, at least 150 times, at least 175 times, at least 200 times, at least 225 times, at least 250 times, at least 275 times, at least 300 times, at least 325 times, at least 350 times, at least 375 times, at least 400 times, at least 425 times, at least 450 times, at least 475 times, at least 500 times, at least 550 times, at least 600 times, at least 650 times, at least 700 times, at least 800 times, at least 900 times, or at least 1,000 times or more per day for at least 2 days, at least a week, at least two weeks, at least one month, at least 3 months, at least 6 months, or at least a year or more. The number of times the steps can be repeated can be accomplished with one or a plurality of reactors 110 operating at the same time. For example, the system 100 can include 2, 3, 4, 5, 6, 7, 8, 9, 10, 11, 12, 13, 14, 15, 16, 17, 18, 19, 20, or more reactors 110 operating at the same time. Any two or more of the plurality of reactors 110 can be operated such that the same process step, e.g., contacting the alkane with the catalyst, can be done at the same time or at a different time with respect to one another.

The system 100 having two or more reactors 110 can include one or more of the control systems 120 for controlling each of the reactors 110. Upon increasing the size of the system 100 by increasing the number of the reactors 110, yields of the dehydrogenated product that includes one or more alkenes also increases. The alkane sources 102, the hydrocarbon fuel source 130, the oxygen-containing fluid source 140, the purge fluid source 150, the reducing gas source 160, and/or the steam source 170 can be increased in size of number of units relative to the number of reactors 110 used in the system 100.

In one or more embodiments, additional disclosure of useful processes and/or systems for producing alkenes from alkanes, decoking processes, and/or regeneration processes can be found in U.S. Pat. Nos. 5,510,557; 6,392,113; 7,842,847; and 8,013,201.

Embodiments of the present disclosure further relate to any one or more of the following paragraphs:

1. A method for dehydrogenating one or more alkanes, comprising: introducing an alkane to a reactor comprising a regenerated catalyst; and contacting the alkane with the regenerated catalyst to produce a dehydrogenated product comprising an alkene and a coked catalyst comprising coke disposed thereon, wherein a pressure within the reactor is less than 38 kPa at the time the alkane is initially introduced to the reactor.

2. The method according to paragraph 1, wherein the regenerated catalyst is at a temperature of about 425° C. to about 800° C. when the alkane is introduced to the reactor.

3. The method according to paragraph 1 or 2, further comprising contacting the coked catalyst with a purge fluid to remove at least a portion of any residual alkane or alkene, wherein the purge fluid comprises steam.

4. The method of according to any one of paragraphs 1 to 3, further comprising contacting the coked catalyst with an oxygen-containing fluid at a temperature of about 600° C. to about 800° C. to produce a decoked catalyst by removing at least a portion of the coke from the coked catalyst.

5. The method according to any one of paragraphs 1 to 3, further comprising contacting the coked catalyst with an oxygen-containing fluid and a hydrocarbon fuel to produce a decoked catalyst, wherein the hydrocarbon fuel is contacted with the coked catalyst for at least 77% of the time that the oxygen-containing fluid is contacted with the coked catalyst.

6. The method according to paragraph 4 or 5, further comprising contacting the decoked catalyst with a reducing gas to produce the regenerated catalyst.

7. The method according to paragraph 6, wherein the reducing gas comprises at least 40 wt % of hydrogen.

8. The method according to paragraph 6, wherein the reducing gas comprises at least 70 wt % of hydrogen.

9. The method according to paragraph 6, wherein the reducing gas comprises at least 90 wt % of hydrogen.

10. The method according to paragraph 6, wherein the reducing gas comprises at least 95 wt % of hydrogen.

11. The method according to paragraph 6, wherein the reducing gas comprises at least 97 wt % of hydrogen.

12. The method according to paragraph 6, wherein the reducing gas comprises at least 99.99 wt % of hydrogen.

13. The method according to any one of paragraphs 1 to 12, wherein the pressure within the reactor is less than 36 kPa at the time the alkane is initially introduced to the reactor.

14. The method according to any one of paragraphs 1 to 12, wherein the pressure within the reactor is less than 33 kPa at the time the alkane is initially introduced to the reactor.

15. The method according to any one of paragraphs 1 to 12, wherein the pressure within the reactor is less than 30 kPa at the time the alkane is initially introduced to the reactor.

16. The method according to any one of paragraphs 1 to 12, wherein the pressure within the reactor is less than 28 kPa at the time the alkane is initially introduced to the reactor.

17. The method according to any one of paragraphs 1 to 12, wherein the pressure within the reactor is less than 25 kPa at the time the alkane is initially introduced to the reactor.

18. The method according to any one of paragraphs 1 to 17, wherein the alkane comprises propane, butane, or a mixture thereof.

19. The method according to any one of paragraphs 1 to 18, wherein the dehydrogenated product comprises propylene, butylene, or a mixture thereof.

20. A method for dehydrogenating one or more alkanes, comprising: introducing an alkane to a reactor comprising a regenerated catalyst; contacting the alkane with the regenerated catalyst to produce a dehydrogenated product comprising an alkene and a coked catalyst comprising coke disposed thereon; contacting the coked catalyst with a purge fluid to remove at least a portion of any residual alkane or alkene; contacting the coked catalyst with an oxygen-containing fluid heated to a temperature of at least 600° C. to produce a decoked catalyst; and contacting the decoked catalyst with a reducing gas to produce the regenerated catalyst, wherein the reducing gas comprises at least 70 wt % of hydrogen.

21. The method of paragraph 19, further comprising contacting the coked catalyst with the oxygen-containing fluid heated to a temperature of about 660° C. to about 800° C. to produce the decoked catalyst.

22. The method according to paragraph 20 or 21, wherein the regenerated catalyst is at a temperature of about 425° C. to about 800° C. when the alkane is contacted therewith.

23. The method according to any one of paragraphs 20 to 22, further comprising contacting the coked catalyst with a hydrocarbon fuel while the coked catalyst is contacted with the oxygen-containing fluid, wherein the hydrocarbon fuel is contacted with the coked catalyst for at least 77% of the time that the oxygen-containing fluid is contacted with the coked catalyst.

24. The method according to any one of paragraphs 20 to 23, wherein the reducing gas comprises at least 90 wt % of hydrogen.

25. The method according to any one of paragraphs 20 to 23, wherein the reducing gas comprises at least 95 wt % of hydrogen.

26. The method according to any one of paragraphs 20 to 23, wherein the reducing gas comprises at least 97 wt % of hydrogen.

27. The method according to any one of paragraphs 20 to 23, wherein the reducing gas comprises at least 99 wt % of hydrogen.

28. The method according to any one of paragraphs 20 to 23, wherein the reducing gas comprises at least 99.9 wt % of hydrogen.

29. The method according to any one of paragraphs 20 to 23, wherein the reducing gas comprises at least 99.99 wt % of hydrogen.

30. The method according to any one of paragraphs 20 to 29, wherein a pressure within the reactor is less than 38 kPa at the time the alkane is initially introduced to the reactor.

31. The method according to any one of paragraphs 20 to 29, wherein a pressure within the reactor is less than 36 kPa at the time the alkane is initially introduced to the reactor.

32. The method according to any one of paragraphs 20 to 29, wherein a pressure within the reactor is less than 33 kPa at the time the alkane is initially introduced to the reactor.

33. The method according to any one of paragraphs 20 to 29, wherein a pressure within the reactor is less than 30 kPa at the time the alkane is initially introduced to the reactor.

34. The method according to any one of paragraphs 20 to 29, wherein a pressure within the reactor is less than 28 kPa at the time the alkane is initially introduced to the reactor.

35. The method according to any one of paragraphs 20 to 34, wherein the alkane comprises propane, butane, or a mixture thereof.

36. The method according to any one of paragraphs 20 to 35, wherein the dehydrogenated product comprises propylene, butylene, or a mixture thereof.

37. A method for dehydrogenating one or more alkanes, comprising: introducing an alkane to a reactor comprising a regenerated catalyst; contacting the alkane with the regenerated catalyst to produce a dehydrogenated product comprising an alkene and a coked catalyst comprising coke disposed thereon; contacting the coked catalyst with steam to remove at least a portion of any residual alkane or alkene; and contacting the coked catalyst with an oxygen-containing fluid and a hydrocarbon fuel to produce a decoked catalyst, wherein the oxygen-containing fluid is at a temperature of at least 600° C., and wherein the hydrocarbon fuel is contacted with the coked catalyst for at least 77% of the time that the oxygen-containing fluid is contacted with the coked catalyst.

38. The method of paragraph 38, wherein the hydrocarbon fuel comprises natural gas.

39. The method of paragraph 37 or 38, wherein the hydrocarbon fuel comprises methane.

40. The method according to any one of paragraphs 37 to 39, wherein the hydrocarbon fuel is contacted with the coked catalyst for at least 80% of the time that the oxygen-containing fluid is contacted with the coked catalyst.

41. The method according to any one of paragraphs 37 to 39, wherein the hydrocarbon fuel is contacted with the coked catalyst for at least 85% of the time that the oxygen-containing fluid is contacted with the coked catalyst.

42. The method according to any one of paragraphs 37 to 39, wherein the hydrocarbon fuel is contacted with the coked catalyst for at least 90% of the time that the oxygen-containing fluid is contacted with the coked catalyst.

43. The method according to any one of paragraphs 37 to 39, wherein the hydrocarbon fuel is contacted with the coked catalyst for at least 98% of the time that the oxygen-containing fluid is contacted with the coked catalyst.

44. The method according to any one of paragraphs 37 to 43, further comprising contacting the decoked catalyst with a reducing gas to produce the regenerated catalyst.

45. The method according to paragraph 44, wherein the reducing gas comprises at least 40 wt % of hydrogen.

46. The method according to paragraph 44, wherein the reducing gas comprises at least 70 wt % of hydrogen.

47. The method according to paragraph 44, wherein the reducing gas comprises at least 90 wt % of hydrogen.

48. The method according to paragraph 44, wherein the reducing gas comprises at least 95 wt % of hydrogen.

49. The method according to paragraph 44, wherein the reducing gas comprises at least 99 wt % of hydrogen.

50. The method according to paragraph 44, wherein the reducing gas comprises at least 99.5 wt % of hydrogen.

51. The method according to any one of paragraphs 37 to 50, wherein a pressure within the reactor is less than 38 kPa at the time the alkane is initially introduced to the reactor.

52. The method according to any one of paragraphs 37 to 50, wherein a pressure within the reactor is less than 36 kPa at the time the alkane is initially introduced to the reactor.

53. The method according to any one of paragraphs 37 to 50, wherein a pressure within the reactor is less than 33 kPa at the time the alkane is initially introduced to the reactor.

54. The method according to any one of paragraphs 37 to 50, wherein a pressure within the reactor is less than 30 kPa at the time the alkane is initially introduced to the reactor.

55. The method according to any one of paragraphs 37 to 50, wherein a pressure within the reactor is less than 28 kPa at the time the alkane is initially introduced to the reactor.

56. The method according to any one of paragraphs 37 to 55, further comprising contacting the coked catalyst with the oxygen-containing fluid heated to a temperature of about 600° C. to about 800° C. to produce the decoked catalyst.

57. The method according to any one of paragraphs 37 to 56, wherein the regenerated catalyst is at a temperature of about 425° C. to about 800° C. when the alkane is contacted therewith.

58. The method according to any one of paragraphs 37 to 57, wherein the alkane comprises propane, butane, or a mixture thereof.

59. The method according to any one of paragraphs 37 to 58, wherein the dehydrogenated product comprises propylene, butylene, or a mixture thereof.

60. A method for dehydrogenating one or more alkanes, comprising: introducing an alkane to a reactor under a vacuum, wherein the reactor comprises a regenerated catalyst; contacting the alkane with the regenerated catalyst to produce a dehydrogenated product comprising an alkene and a coked catalyst comprising coke disposed thereon; contacting the coked catalyst with steam to remove at least a portion of any residual alkane or alkene; contacting the coked catalyst with an oxygen-containing fluid heated to a temperature of at least 600° C. to produce a decoked catalyst; introducing a reducing gas to the reactor to produce the regenerated catalyst; and stopping introduction of the reducing gas to the reactor at least 10 seconds prior to re-introducing the alkane to the reactor.

61. The method according to paragraph 60, further comprising contacting the coked catalyst with a hydrocarbon fuel to produce the decoked catalyst, wherein the hydrocarbon fuel comprises natural gas.

62. The method according to paragraph 60 or 61, further comprising contacting the coked catalyst with a hydrocarbon fuel to produce the decoked catalyst, wherein the hydrocarbon fuel comprises methane.

63. The method according to paragraph 61 or 62, wherein the hydrocarbon fuel is contacted with the coked catalyst for at least 77% of the time that the oxygen-containing fluid is contacted with the coked catalyst.

64. The method according to paragraph 61 or 62, wherein the hydrocarbon fuel is contacted with the coked catalyst for at least 80% of the time that the oxygen-containing fluid is contacted with the coked catalyst.

65. The method according to paragraph 61 or 62, wherein the hydrocarbon fuel is contacted with the coked catalyst for at least 85% of the time that the oxygen-containing fluid is contacted with the coked catalyst.

66. The method according to paragraph 61 or 62, wherein the hydrocarbon fuel is contacted with the coked catalyst for at least 90% of the time that the oxygen-containing fluid is contacted with the coked catalyst.

67. The method according to paragraph 61 or 62, wherein the hydrocarbon fuel is contacted with the coked catalyst for at least 95% of the time that the oxygen-containing fluid is contacted with the coked catalyst.

68. The method according to any one of paragraphs 60 to 67, wherein the reducing gas comprises at least 70 wt % of hydrogen.

69. The method according to any one of paragraphs 60 to 67, wherein the reducing gas comprises at least 90 wt % of hydrogen.

70. The method according to any one of paragraphs 60 to 67, wherein the reducing gas comprises at least 95 wt % of hydrogen.

71. The method according to any one of paragraphs 60 to 67, wherein the reducing gas comprises at least 97 wt % of hydrogen.

72. The method according to any one of paragraphs 60 to 67, wherein the reducing gas comprises at least 99 wt % of hydrogen.

73. The method according to any one of paragraphs 60 to 67, wherein the reducing gas comprises at least 99.5 wt % of hydrogen.

74. The method according to any one of paragraphs 60 to 73, wherein a pressure within the reactor is less than 38 kPa at the time the alkane is initially introduced to the reactor.

75. The method according to any one of paragraphs 60 to 73, wherein a pressure within the reactor is less than 36 kPa at the time the alkane is initially introduced to the reactor.

76. The method according to any one of paragraphs 60 to 73, wherein a pressure within the reactor is less than 33 kPa at the time the alkane is initially introduced to the reactor.

77. The method according to any one of paragraphs 60 to 73, wherein a pressure within the reactor is less than 30 kPa at the time the alkane is initially introduced to the reactor.

78. The method according to any one of paragraphs 60 to 73, wherein a pressure within the reactor is less than 28 kPa at the time the alkane is initially introduced to the reactor.

79. The method according to any one of paragraphs 60 to 78, further comprising contacting the coked catalyst with the oxygen-containing fluid heated to a temperature of about 600° C. to about 800° C. to produce the decoked catalyst.

80. The method according to any one of paragraphs 60 to 79, wherein the regenerated catalyst is at a temperature of about 425° C. to about 800° C. when the alkane is contacted therewith.

81. The method according to any one of paragraphs 60 to 80, wherein the alkane comprises propane, butane, or a mixture thereof.

82. The method according to any one of paragraphs 60 to 81, wherein the dehydrogenated product comprises propylene, butylene, or a mixture thereof.

83. The method according to any one of paragraphs 60 to 82, wherein introduction of the reducing gas to the reactor stops at least 15 seconds prior to re-introducing the alkane to the reactor.

84. The method according to any one of paragraphs 60 to 82, wherein introduction of the reducing gas to the reactor stops at least 20 seconds prior to re-introducing the alkane to the reactor.

85. The method according to any one of paragraphs 60 to 82, wherein introduction of the reducing gas to the reactor stops at least 25 seconds prior to re-introducing the alkane to the reactor.

86. A method for dehydrogenating one or more alkanes, comprising: introducing an alkane to a reactor under a vacuum, wherein the reactor comprises a regenerated catalyst; contacting the alkane with the regenerated catalyst to produce a dehydrogenated product comprising an alkene and a coked catalyst comprising coke disposed thereon; contacting the coked catalyst with steam to remove at least a portion of any residual alkane or alkene; contacting the coked catalyst with an oxygen-containing fluid heated to a temperature of at least 600° C. to produce a decoked catalyst; introducing a reducing gas to the reactor to produce the regenerated catalyst; recovering an off-gas from the reactor while the reducing gas is introduced to the reactor; venting the off-gas directly to the atmosphere without passing through a waste heat boiler.

87. The method according to paragraph 86, further comprising contacting the coked catalyst with a hydrocarbon fuel, wherein the hydrocarbon fuel comprises natural gas.

88. The method according to paragraph 86 or 87, further comprising contacting the coked catalyst with a hydrocarbon fuel, wherein the hydrocarbon fuel comprises methane.

89. The method according to paragraph 86, further comprising contacting the coked catalyst with a hydrocarbon fuel, wherein the hydrocarbon fuel is contacted with the coked catalyst for at least 80% of the time that the oxygen-containing fluid is contacted with the coked catalyst.

90. The method according to paragraph 86, further comprising contacting the coked catalyst with a hydrocarbon fuel, wherein the hydrocarbon fuel is contacted with the coked catalyst for at least 85% of the time that the oxygen-containing fluid is contacted with the coked catalyst.

91. The method according to paragraph 86, further comprising contacting the coked catalyst with a hydrocarbon fuel, wherein the hydrocarbon fuel is contacted with the coked catalyst for at least 90% of the time that the oxygen-containing fluid is contacted with the coked catalyst.

92. The method according to paragraph 86, further comprising contacting the coked catalyst with a hydrocarbon fuel, wherein the hydrocarbon fuel is contacted with the coked catalyst for at least 95% of the time that the oxygen-containing fluid is contacted with the coked catalyst.

93. The method according to paragraph 86, further comprising contacting the coked catalyst with a hydrocarbon fuel, wherein the hydrocarbon fuel is contacted with the coked catalyst for at least 98% of the time that the oxygen-containing fluid is contacted with the coked catalyst.

94. The method according to any one of paragraphs 86 to 93, wherein the reducing gas comprises at least 90 wt % of hydrogen.

95. The method according to any one of paragraphs 86 to 93, wherein the reducing gas comprises at least 93 wt % of hydrogen.

96. The method according to any one of paragraphs 86 to 93, wherein the reducing gas comprises at least 95 wt % of hydrogen.

97. The method according to any one of paragraphs 86 to 93, wherein the reducing gas comprises at least 97 wt % of hydrogen.

98. The method according to any one of paragraphs 86 to 93, wherein the reducing gas comprises at least 99 wt % of hydrogen.

99. The method according to any one of paragraphs 86 to 93, wherein the reducing gas comprises at least 99.5 wt % of hydrogen.

100. The method according to any one of paragraphs 86 to 99, wherein a pressure within the reactor is less than 38 kPa at the time the alkane is initially introduced to the reactor.

101. The method according to any one of paragraphs 86 to 99, wherein a pressure within the reactor is less than 36 kPa at the time the alkane is initially introduced to the reactor.

102. The method according to any one of paragraphs 86 to 99, wherein a pressure within the reactor is less than 33 kPa at the time the alkane is initially introduced to the reactor.

103. The method according to any one of paragraphs 86 to 99, wherein a pressure within the reactor is less than 30 kPa at the time the alkane is initially introduced to the reactor.

104. The method according to any one of paragraphs 86 to 99, wherein a pressure within the reactor is less than 28 kPa at the time the alkane is initially introduced to the reactor.

105. The method according to any one of paragraphs 86 to 104, wherein the regenerated catalyst is at a temperature of about 425° C. to about 800° C. when the alkane is contacted therewith.

106. The method according to any one of paragraphs 86 to 105, wherein the alkane comprises propane, butane, or a mixture thereof.

107. The method according to any one of paragraphs 86 to 105, wherein the dehydrogenated product comprises propylene, butylene, or a mixture thereof.

108. The method according to any one of paragraphs 86 to 105, wherein the introducing of the alkane to the reactor, the contacting the alkane with the regenerated catalyst, the contacting the coked catalyst with steam, the contacting the coked catalyst with the oxygen-containing fluid, the introducing of the reducing gas to the reactor, the recovering of the off-gas from the reactor, and the venting the off-gas directly to the atmosphere are repeated a plurality of times.

109. The method according to any one of paragraphs 86 to 105, wherein the introducing of the alkane to the reactor, the contacting the alkane with the regenerated catalyst, the contacting the coked catalyst with steam, the contacting the coked catalyst with the oxygen-containing fluid, the introducing of the reducing gas to the reactor, the recovering of the off-gas from the reactor, and the venting the off-gas directly to the atmosphere are repeated a plurality of times to provide a continuous process.

110. The method according to any one of paragraphs 86 to 105, wherein the introducing of the alkane to the reactor, the contacting the alkane with the regenerated catalyst, the contacting the coked catalyst with steam, the contacting the coked catalyst with the oxygen-containing fluid, the introducing of the reducing gas to the reactor, the recovering of the off-gas from the reactor, and the venting the off-gas directly to the atmosphere are sequentially repeated a plurality of times to provide a continuous process.

111. The method according to any one of paragraphs 86 to 105, wherein the introducing of the alkane to the reactor, the contacting the alkane with the regenerated catalyst, the contacting the coked catalyst with steam, the contacting the coked catalyst with the oxygen-containing fluid, the introducing of the reducing gas to the reactor, the recovering of the off-gas from the reactor, and the venting the off-gas directly to the atmosphere are repeated at least 100 times.

112. The method according to any one of paragraphs 86 to 105, wherein the introducing of the alkane to the reactor, the contacting the alkane with the regenerated catalyst, the contacting the coked catalyst with steam, the contacting the coked catalyst with the oxygen-containing fluid, the introducing of the reducing gas to the reactor, the recovering of the off-gas from the reactor, and the venting the off-gas directly to the atmosphere are repeated at least 100 times to provide a continuous process.

113. The method according to any one of paragraphs 86 to 105, wherein the introducing of the alkane to the reactor, the contacting the alkane with the regenerated catalyst, the contacting the coked catalyst with steam, the contacting the coked catalyst with the oxygen-containing fluid, the introducing of the reducing gas to the reactor, the recovering of the off-gas from the reactor, and the venting the off-gas directly to the atmosphere are sequentially repeated at least 100 times to provide a continuous process.

114. The method according to any one of paragraphs 86 to 105, wherein the introducing of the alkane to the reactor, the contacting the alkane with the regenerated catalyst, the contacting the coked catalyst with steam, the contacting the coked catalyst with the oxygen-containing fluid, the introducing of the reducing gas to the reactor, the recovering of the off-gas from the reactor, and the venting the off-gas directly to the atmosphere are repeated at least 500 times.

115. The method according to any one of paragraphs 86 to 105, wherein the introducing of the alkane to the reactor, the contacting the alkane with the regenerated catalyst, the contacting the coked catalyst with steam, the contacting the coked catalyst with the oxygen-containing fluid, the introducing of the reducing gas to the reactor, the recovering of the off-gas from the reactor, and the venting the off-gas directly to the atmosphere are repeated at least 500 times to provide a continuous process.

116. The method according to any one of paragraphs 86 to 105, wherein the introducing of the alkane to the reactor, the contacting the alkane with the regenerated catalyst, the contacting the coked catalyst with steam, the contacting the coked catalyst with the oxygen-containing fluid, the introducing of the reducing gas to the reactor, the recovering of the off-gas from the reactor, and the venting the off-gas directly to the atmosphere are sequentially repeated at least 500 times to provide a continuous process.

117. The method according to any one of paragraphs 86 to 105, wherein the introducing of the alkane to the reactor, the contacting the alkane with the regenerated catalyst, the contacting the coked catalyst with steam, the contacting the coked catalyst with the oxygen-containing fluid, the introducing of the reducing gas to the reactor, the recovering of the off-gas from the reactor, and the venting the off-gas directly to the atmosphere are repeated at least 5 times a day for at least 2 days.

118. The method according to any one of paragraphs 86 to 105, wherein the introducing of the alkane to the reactor, the contacting the alkane with the regenerated catalyst, the contacting the coked catalyst with steam, the contacting the coked catalyst with the oxygen-containing fluid, the introducing of the reducing gas to the reactor, the recovering of the off-gas from the reactor, and the venting the off-gas directly to the atmosphere are sequentially repeated at least 5 times a day for at least 2 days.

119. The method according to any one of paragraphs 86 to 105, wherein the introducing of the alkane to the reactor, the contacting the alkane with the regenerated catalyst, the contacting the coked catalyst with steam, the contacting the coked catalyst with the oxygen-containing fluid, the introducing of the reducing gas to the reactor, the recovering of the off-gas from the reactor, and the venting the off-gas directly to the atmosphere are repeated at least 25 times a day for at least 2 days.

120. The method according to any one of paragraphs 86 to 105, wherein the introducing of the alkane to the reactor, the contacting the alkane with the regenerated catalyst, the contacting the coked catalyst with steam, the contacting the coked catalyst with the oxygen-containing fluid, the introducing of the reducing gas to the reactor, the recovering of the off-gas from the reactor, and the venting the off-gas directly to the atmosphere are sequentially repeated at least 25 times a day for at least 2 days.

121. The method according to any one of paragraphs 86 to 105, wherein the introducing of the alkane to the reactor, the contacting the alkane with the regenerated catalyst, the contacting the coked catalyst with steam, the contacting the coked catalyst with the oxygen-containing fluid, the introducing of the reducing gas to the reactor, the recovering of the off-gas from the reactor, and the venting the off-gas directly to the atmosphere are repeated at least 50 times a day for at least 2 days.

122. The method according to any one of paragraphs 86 to 105, wherein the introducing of the alkane to the reactor, the contacting the alkane with the regenerated catalyst, the contacting the coked catalyst with steam, the contacting the coked catalyst with the oxygen-containing fluid, the introducing of the reducing gas to the reactor, the recovering of the off-gas from the reactor, and the venting the off-gas directly to the atmosphere are sequentially repeated at least 50 times a day for at least 2 days.

123. A method for dehydrogenating one or more alkanes, comprising: introducing an alkane to a reactor under a vacuum, wherein the reactor comprises a regenerated catalyst; contacting the alkane with the regenerated catalyst to produce a dehydrogenated product comprising an alkene and a coked catalyst comprising coke disposed thereon; contacting the coked catalyst with steam to remove at least a portion of any residual alkane or alkene; contacting the coked catalyst with an oxygen-containing fluid heated to a temperature of at least 600° C. to produce a decoked catalyst; introducing a reducing gas to the reactor to produce the regenerated catalyst; and stopping introduction of the reducing gas to the reactor at least 28 seconds prior to re-introducing the alkane to the reactor.

124. The method according to paragraph 123, further comprising contacting the coked catalyst with a hydrocarbon fuel to produce the decoked catalyst, wherein the hydrocarbon fuel comprises natural gas.

125. The method according to paragraph 123 or 124, further comprising contacting the coked catalyst with a hydrocarbon fuel to produce the decoked catalyst, wherein the hydrocarbon fuel comprises methane.

126. The method according to paragraph 123 or 124, wherein the hydrocarbon fuel is contacted with the coked catalyst for at least 77% of the time that the oxygen-containing fluid is contacted with the coked catalyst.

127. The method according to paragraph 123 or 124, wherein the hydrocarbon fuel is contacted with the coked catalyst for at least 80% of the time that the oxygen-containing fluid is contacted with the coked catalyst.

128. The method according to paragraph 123 or 124, wherein the hydrocarbon fuel is contacted with the coked catalyst for at least 85% of the time that the oxygen-containing fluid is contacted with the coked catalyst.

129. The method according to paragraph 123 or 124, wherein the hydrocarbon fuel is contacted with the coked catalyst for at least 90% of the time that the oxygen-containing fluid is contacted with the coked catalyst.

130. The method according to paragraph 123 or 124, wherein the hydrocarbon fuel is contacted with the coked catalyst for at least 95% of the time that the oxygen-containing fluid is contacted with the coked catalyst.

131. The method according to any one of paragraphs 123 to 130, wherein the reducing gas comprises at least 70 wt % of hydrogen.

132. The method according to any one of paragraphs 123 to 130, wherein the reducing gas comprises at least 90 wt % of hydrogen.

133. The method according to any one of paragraphs 123 to 130, wherein the reducing gas comprises at least 95 wt % of hydrogen.

134. The method according to any one of paragraphs 123 to 130, wherein the reducing gas comprises at least 97 wt % of hydrogen.

135. The method according to any one of paragraphs 123 to 130, wherein the reducing gas comprises at least 99 wt % of hydrogen.

136. The method according to any one of paragraphs 123 to 130, wherein the reducing gas comprises at least 99.5 wt % of hydrogen.

137. The method according to any one of paragraphs 123 to 136, wherein a pressure within the reactor is less than 38 kPa at the time the alkane is initially introduced to the reactor.

138. The method according to any one of paragraphs 123 to 136, wherein a pressure within the reactor is less than 36 kPa at the time the alkane is initially introduced to the reactor.

139. The method according to any one of paragraphs 123 to 136, wherein a pressure within the reactor is less than 33 kPa at the time the alkane is initially introduced to the reactor.

140. The method according to any one of paragraphs 123 to 136, wherein a pressure within the reactor is less than 30 kPa at the time the alkane is initially introduced to the reactor.

141. The method according to any one of paragraphs 123 to 136, wherein a pressure within the reactor is less than 28 kPa at the time the alkane is initially introduced to the reactor.

142. The method according to any one of paragraphs 123 to 140, further comprising contacting the coked catalyst with the oxygen-containing fluid heated to a temperature of about 600° C. to about 800° C. to produce the decoked catalyst.

143. The method according to any one of paragraphs 123 to 142, wherein the regenerated catalyst is at a temperature of about 425° C. to about 800° C. when the alkane is contacted therewith.

144. The method according to any one of paragraphs 123 to 143, wherein the alkane comprises propane, butane, or a mixture thereof.

145. The method according to any one of paragraphs 123 to 144, wherein the dehydrogenated product comprises propylene, butylene, or a mixture thereof.

146. The method according to any one of paragraphs 123 to 145, wherein introduction of the reducing gas to the reactor stops at least 35 seconds prior to re-introducing the alkane to the reactor.

147. The method according to any one of paragraphs 123 to 146, wherein introduction of the reducing gas to the reactor stops at least 40 seconds prior to re-introducing the alkane to the reactor.

148. The method according to any one of paragraphs 123 to 147, wherein introduction of the reducing gas to the reactor stops at least 50 seconds prior to re-introducing the alkane to the reactor.

149. A method for dehydrogenating one or more alkanes, comprising: introducing an alkane to a reactor under a vacuum, wherein the reactor comprises a regenerated catalyst; contacting the alkane with the regenerated catalyst to produce a dehydrogenated product comprising an alkene and a coked catalyst comprising coke disposed thereon; contacting the coked catalyst with steam to remove at least a portion of any residual alkane or alkene; contacting the coked catalyst with an oxygen-containing fluid heated to a temperature of at least 600° C. to produce a decoked catalyst; introducing a reducing gas to the reactor while evacuating the reactor to produce the regenerated catalyst; stopping introduction of the reducing gas to the reactor while maintaining the evacuation of the reactor; stopping the evacuation of the reactor at least 10 seconds after stopping the introduction of the reducing gas; and introducing additional alkane to the reactor.

150. A method for dehydrogenating propane, comprising: contacting propane with a regenerated catalyst within a process environment to produce propylene and a coked catalyst comprising coke disposed thereon, wherein the process environment is at a pressure of about 20 kPa to about 38 kPa and at a temperature of about 425° C. to about 800° C.; removing at least a portion of the propylene from the process environment; removing at least a portion of any residual propane or propylene from the process environment comprising the coked catalyst; contacting the coked catalyst with an oxygen-containing fluid within the process environment to produce a decoked catalyst, wherein the process environment is at a temperature of about 425° C. to about 800° C.; contacting the decoked catalyst with a reducing gas within the process environment to produce the regenerated catalyst; stopping contact of the reducing gas with the decoked catalyst within the process environment; removing at least a portion of any residual reducing gas from the process environment comprising the regenerated catalyst by exposing the process environment to a vacuum for at least 10 seconds after stopping contact of the reducing gas with the decoked catalyst; and contacting additional propane with the regenerated catalyst within the process environment to produce additional propylene and additional coked catalyst comprising coke disposed thereon.

151. The method according to paragraph 149, wherein the reducing gas comprises at least 70 wt % of hydrogen (H₂), and wherein the decoked catalyst is contacted with reducing gas for less than 110 seconds.

152. The method according to paragraph 149, wherein the reducing gas comprises at least 70 wt % of hydrogen (H₂), and wherein the decoked catalyst is contacted with reducing gas for less than 100 seconds.

153. The method according to paragraph 149, wherein the reducing gas comprises at least 70 wt % of hydrogen (H₂), and wherein the decoked catalyst is contacted with reducing gas for less than 90 seconds.

154. The method according to paragraph 149, wherein the reducing gas comprises at least 70 wt % of hydrogen (H₂), and wherein the decoked catalyst is contacted with reducing gas for about 75 seconds to about 100 seconds.

155. The method according to paragraph 149, wherein the reducing gas comprises at least 70 wt % of hydrogen (H₂), and wherein the decoked catalyst is contacted with reducing gas for about 60 seconds to about 90 seconds.

156. The method according to paragraph 149, wherein the reducing gas comprises at least 70 wt % of hydrogen (H₂), and wherein the decoked catalyst is contacted with reducing gas for about 80 seconds to about 90 seconds.

157. The method according to any one of paragraphs 149 to 156, further comprising: closing a valve to cease exposure of the process environment to the vacuum; and contacting additional propane to the regenerated catalyst within the process environment to produce additional propylene.

158. The method of according to any one of paragraphs 149 to 157, wherein the at least a portion of any residual propane or propylene is removed from the process environment by exposing the process environment to a purge gas, a vacuum, or a combination of the purge gas and the vacuum.

159. The method according to any one of paragraphs 149 to 158, wherein the process environment is at a pressure of about 20 kPa to about 29 kPa and at a temperature of about 425° C. to about 800° C. when the propane is contacted with the regenerated catalyst.

160. A method for dehydrogenating one or more alkanes, comprising: contacting a catalyst with an alkane to produce a coked catalyst comprising coke disposed thereon and a dehydrogenated product comprising an alkene, wherein the alkane is contacted with the catalyst under a pressure of less than 101 kPa; separating the dehydrogenated product from the coked catalyst; contacting the coked catalyst with a purge fluid to remove at least a portion of any residual alkane, any residual alkene, or a combination thereof from the coked catalyst; contacting the coked catalyst with an oxygen-containing fluid; combusting at least a portion of the coke disposed on the catalyst in the presence of the oxygen-containing fluid to produce a decoked catalyst; contacting the decoked catalyst with a reducing gas to produce a regenerated catalyst and an off-gas; and contacting the regenerated catalyst with additional alkane to produce additional dehydrogenated product and additional coked catalyst.

161. The method according to paragraph 160, wherein the alkane is contacted with the catalyst at a pressure of less than 38 kPa.

162. The method according to paragraph 160, wherein the alkane is contacted with the catalyst at a pressure of about 20 kPa to about 38 kPa.

163. The method according to any one of paragraphs 160 to 162, wherein contact of the decoked catalyst with the reducing gas is stopped at least 10 seconds prior to contacting the additional alkane with the regenerated catalyst.

164. The method according to any one of paragraphs 160 to 163, further comprising introducing the off-gas directly to the atmosphere without passing the off-gas through a waste heat boiler.

165. The method according to any one of paragraphs 160 to 164, further comprising combusting a hydrocarbon fuel in the presence of the coked catalyst and the oxygen-containing fluid when the coke disposed on the catalyst is combusted in the presence of the oxygen-containing fluid, wherein the hydrocarbon fuel is combusted for at least 77% of the time the coke disposed on the catalyst is combusted in the presence of the oxygen-containing fluid.

166. The method according to any one of paragraphs 160 to 165, wherein: the alkane comprises propane, butane, or a mixture thereof, the catalyst comprises platinum, palladium, gold, indium, chromium, tin, iron, molybdenum, tungsten, nickel, silver, any alloy thereof, any oxide thereof, or any mixture thereof, the alkene comprises propene, butylene, or a mixture thereof, the purge fluid comprises steam, the oxygen-containing fluid comprises air, and the hydrocarbon fuel comprises methane.

167. The method according to any one of paragraphs 160 to 166, further comprising: introducing the off-gas directly to the atmosphere without passing the off-gas through a waste heat boiler; and combusting a hydrocarbon fuel in the presence of the coked catalyst and the oxygen-containing fluid when the coke disposed on the catalyst is combusted in the presence of the oxygen-containing fluid, wherein the hydrocarbon fuel is combusted for at least 90% of the time the coke disposed on the catalyst is combusted in the presence of the oxygen-containing fluid.

168. The method according to any one of paragraphs 160 to 167, wherein the catalyst is at a temperature of about 425° C. to about 800° C. when contacted with the alkane.

169. The method according to any one of paragraphs 160 to 168, wherein the oxygen-containing fluid is at a temperature of at least 600° C. to about 800° C. when contacted with the coked catalyst.

170. A method for dehydrogenating one or more alkanes, comprising: introducing an alkane to a reactor that comprises a catalyst disposed therein; contacting the catalyst with the alkane to produce a coked catalyst comprising coke disposed thereon and a dehydrogenated product comprising an alkene, wherein the alkane is contacted with the catalyst under a pressure of less than 101 kPa; removing the dehydrogenated product from the reactor; contacting the coked catalyst with a purge fluid to remove at least a portion of any residual alkane, any residual alkene, or a combination thereof from the coked catalyst; contacting the coked catalyst with an oxygen-containing fluid heated to a temperature of at least 600° C. to about 800° C.; combusting at least a portion of the coke disposed on the catalyst in the presence of the oxygen-containing fluid to produce a decoked catalyst; contacting the decoked catalyst with a reducing gas to produce a regenerated catalyst; introducing additional alkane to the reactor that comprises the regenerated catalyst, wherein contact of the decoked catalyst with the reducing gas is stopped at least 10 seconds prior to introducing the additional alkane to the reactor; and contacting the additional alkane with the regenerated catalyst to produce additional dehydrogenated product and additional coked catalyst.

171. The method according to paragraph 170, wherein the alkane is contacted with the catalyst under a pressure of about 20 kPa to less than 38 kPa.

172. The method according to paragraph 170 or 171, wherein contact of the decoked catalyst with the reducing gas is stopped at least 28 seconds prior to introducing the additional alkane to the reactor.

173. The method according to any one of paragraphs 170 to 172, wherein the catalyst is at a temperature of about 425° C. to about 800° C. when contacted with the alkane, and wherein the oxygen-containing fluid is at a temperature of about 660° C. to about 800° C. when contacted with the coked catalyst.

174. The method according to any one of paragraphs 170 to 173, wherein: the alkane comprises propane, butane, or a mixture thereof, the catalyst comprises platinum, palladium, gold, indium, chromium, tin, iron, molybdenum, tungsten, nickel, silver, any alloy thereof, any oxide thereof, or any mixture thereof, the alkene comprises propene, butylene, or a mixture thereof, the purge fluid comprises steam, and the oxygen-containing fluid comprises air.

175. The method according to any one of paragraphs 170 to 174, further comprising combusting a hydrocarbon fuel in the presence of the coked catalyst and the oxygen-containing fluid when the coke disposed on the catalyst is combusted in the presence of the oxygen-containing fluid, wherein the hydrocarbon fuel is combusted for at least 77% of the time the coke disposed on the catalyst is combusted in the presence of the oxygen-containing fluid, and wherein the hydrocarbon fuel comprises methane.

176. A method for dehydrogenating propane, comprising: contacting a catalyst with propane within a process environment to produce propylene and a coked catalyst comprising coke disposed thereon, wherein the process environment is at a pressure of about 20 kPa to about 38 kPa, the propane is at a temperature of about 400° C. to about 650° C., and the regenerated catalyst is at a temperature of about 425° C. to about 750° C. when the propane is contacted with the regenerated catalyst; removing at least a portion of the propylene from the process environment; contacting the coked catalyst with a purge fluid to remove at least a portion of any residual propane, at least a portion of any residual propylene, or both from the process environment comprising the coked catalyst, wherein the purge fluid is at a temperature of about 120° C. to about 600° C.; contacting the coked catalyst with an oxygen-containing fluid within the process environment to combust at least a portion of the coke disposed thereon and produce a decoked catalyst, wherein the oxygen-containing fluid is at a temperature of about 500° C. to about 800° C. when the coked catalyst is contacted with the oxygen-containing fluid; contacting the decoked catalyst with a reducing gas within the process environment to produce a regenerated catalyst; stopping contact of the reducing gas with the regenerated catalyst within the process environment; removing at least a portion of any residual reducing gas from the process environment comprising the regenerated catalyst by exposing the process environment to a vacuum for at least 10 seconds after stopping contact of the reducing gas with the regenerated catalyst; and contacting additional propane with the regenerated catalyst within the process environment to produce additional propylene and additional coked catalyst comprising coke disposed thereon.

177. The method according to paragraph 176, wherein the reducing gas comprises at least 70 wt % of hydrogen, and wherein the decoked catalyst is contacted with the reducing gas for less than 110 seconds.

178. The method according to paragraph 176 or 177, wherein the process environment is at a pressure of about 20 kPa to about 29 kPa when the propane is contacted with the regenerated catalyst.

179. The method according to any one of paragraphs 176 to 177, further comprising contacting the coked catalyst with a hydrocarbon fuel within the process environment when the cocked catalyst is contacted with the oxygen-containing fluid, wherein the coked catalyst is contacted with the hydrocarbon fuel for at least 77% of the time the coked catalyst is contacted with the oxygen-containing fluid.

180. The method according to any one of paragraphs 160 to 179, wherein the reducing as comprises at least 40 wt % of hydrogen.

181. The method according to any one of paragraphs 160 to 180, wherein the reducing as comprises at least 90 wt % of hydrogen.

182. The method according to any one of paragraphs 160 to 181, wherein the reducing as comprises at least 97 wt % of hydrogen.

183. The method according to any one of paragraphs 160 to 182, wherein the reducing as comprises at least 99.99 wt % of hydrogen.

184. The method according to any one of paragraphs 160 to 183, wherein the alkane is contacted with the catalyst under a pressure of less than 36 kPa.

185. The method according to any one of paragraphs 160 to 184, wherein the alkane is contacted with the catalyst under a pressure of less than 33 kPa.

186. The method according to any one of paragraphs 160 to 185, wherein the alkane is contacted with the catalyst under a pressure of less than 28 kPa.

187. The method according to any one of paragraphs 160 to 186, wherein the alkane is contacted with the catalyst under a pressure of less than 25 kPa.

188. The method according to any one of paragraphs 160 to 187, wherein the alkane comprises propane, butane, or a mixture thereof.

189. The method according to any one of paragraphs 160 to 188, wherein the catalyst comprises platinum, palladium, gold, indium, chromium, tin, iron, molybdenum, tungsten, nickel, silver, any alloy thereof, any oxide thereof, or any mixture thereof.

190. The method according to any one of paragraphs 160 to 189, wherein the alkene comprises propene, butylene, or a mixture thereof.

191. The method according to any one of paragraphs 160 to 190, wherein the purge fluid comprises steam.

192. The method according to any one of paragraphs 160 to 191, wherein the oxygen-containing fluid comprises air.

193. The method according to any one of paragraphs 165 to 169, 175, or 179 to 192, wherein the hydrocarbon fuel comprises methane.

194. The method according to any one of paragraphs 165 to 169, 175, or 179 to 192, wherein the hydrocarbon fuel comprises natural gas.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

What is claimed is:

1. A method for dehydrogenating one or more alkanes, comprising:
contacting a catalyst with an alkane to produce a coked catalyst comprising coke disposed thereon and a dehydrogenated product comprising an alkene, wherein the alkane is contacted with the catalyst under a pressure of less than 101 kPa;
separating the dehydrogenated product from the coked catalyst;
contacting the coked catalyst with a purge fluid to remove at least a portion of any residual alkane, any residual alkene, or a combination thereof from the coked catalyst;
contacting the coked catalyst with an oxygen-containing fluid;
combusting at least a portion of the coke disposed on the catalyst in the presence of the oxygen-containing fluid to produce a decoked catalyst;
contacting the decoked catalyst with a reducing gas to produce a regenerated catalyst and an off-gas; and
contacting the regenerated catalyst with additional alkane to produce additional dehydrogenated product and additional coked catalyst.

2. The method of claim 1, wherein the alkane is contacted with the catalyst at a pressure of less than 38 kPa.

3. The method of claim 1, wherein the alkane is contacted with the catalyst at a pressure of about 20 kPa to about 38 kPa.

4. The method of claim 1, wherein contact of the decoked catalyst with the reducing gas is stopped at least 10 seconds prior to contacting the additional alkane with the regenerated catalyst.

5. The method of claim 1, further comprising introducing the off-gas directly to the atmosphere without passing the off-gas through a waste heat boiler.

6. The method of claim 1, further comprising combusting a hydrocarbon fuel in the presence of the coked catalyst and the oxygen-containing fluid when the coke disposed on the catalyst is combusted in the presence of the oxygen-containing fluid, wherein the hydrocarbon fuel is combusted for at least 77% of the time the coke disposed on the catalyst is combusted in the presence of the oxygen-containing fluid.

7. The method of claim 6, wherein:
the alkane comprises propane, butane, or a mixture thereof,
the catalyst comprises platinum, palladium, gold, indium, chromium, tin, iron, molybdenum, tungsten, nickel, silver, any alloy thereof, any oxide thereof, or any mixture thereof,
the alkene comprises propene, butylene, or a mixture thereof,
the purge fluid comprises steam,
the oxygen-containing fluid comprises air, and
the hydrocarbon fuel comprises methane.

8. The method of claim 1, further comprising:
introducing the off-gas directly to the atmosphere without passing the off-gas through a waste heat boiler; and
combusting a hydrocarbon fuel in the presence of the coked catalyst and the oxygen-containing fluid when the coke disposed on the catalyst is combusted in the presence of the oxygen-containing fluid, wherein the hydrocarbon fuel is combusted for at least 90% of the time the coke disposed on the catalyst is combusted in the presence of the oxygen-containing fluid.

9. The method of claim 1, wherein the catalyst is at a temperature of about 425° C. to about 800° C. when contacted with the alkane.

10. The method of claim 1, wherein the oxygen-containing fluid is at a temperature of at least 600° C. to about 800° C. when contacted with the coked catalyst.

11. A method for dehydrogenating one or more alkanes, comprising:
introducing an alkane to a reactor that comprises a catalyst disposed therein;
contacting the catalyst with the alkane to produce a coked catalyst comprising coke disposed thereon and a dehydrogenated product comprising an alkene, wherein the alkane is contacted with the catalyst under a pressure of less than 101 kPa;
removing the dehydrogenated product from the reactor;
contacting the coked catalyst with a purge fluid to remove at least a portion of any residual alkane, any residual alkene, or a combination thereof from the coked catalyst;
contacting the coked catalyst with an oxygen-containing fluid heated to a temperature of at least 600° C. to about 800° C.;
combusting at least a portion of the coke disposed on the catalyst in the presence of the oxygen-containing fluid to produce a decoked catalyst;
contacting the decoked catalyst with a reducing gas to produce a regenerated catalyst;
introducing additional alkane to the reactor that comprises the regenerated catalyst, wherein contact of the decoked catalyst with the reducing gas is stopped at least 10 seconds prior to introducing the additional alkane to the reactor; and
contacting the additional alkane with the regenerated catalyst to produce additional dehydrogenated product and additional coked catalyst.

12. The method of claim 11, wherein the alkane is contacted with the catalyst under a pressure of about 20 kPa to less than 38 kPa.

13. The method of claim 11, wherein contact of the decoked catalyst with the reducing gas is stopped at least 28 seconds prior to introducing the additional alkane to the reactor.

14. The method of claim 11, wherein the catalyst is at a temperature of about 425° C. to about 800° C. when contacted with the alkane, and wherein the oxygen-containing fluid is at a temperature of about 660° C. to about 800° C. when contacted with the coked catalyst.

15. The method of claim 11, wherein:
the alkane comprises propane, butane, or a mixture thereof,
the catalyst comprises platinum, palladium, gold, indium, chromium, tin, iron, molybdenum, tungsten, nickel, silver, any alloy thereof, any oxide thereof, or any mixture thereof,
the alkene comprises propene, butylene, or a mixture thereof,
the purge fluid comprises steam, and
the oxygen-containing fluid comprises air.

16. The method of claim 11, further comprising combusting a hydrocarbon fuel in the presence of the coked catalyst and the oxygen-containing fluid when the coke disposed on the catalyst is combusted in the presence of the oxygen-containing fluid, wherein the hydrocarbon fuel is combusted for at least 77% of the time the coke disposed on the catalyst is combusted in the presence of the oxygen-containing fluid, and wherein the hydrocarbon fuel comprises methane.

17. A method for dehydrogenating propane, comprising:
contacting a catalyst with propane within a process environment to produce propylene and a coked catalyst comprising coke disposed thereon, wherein the process environment is at a pressure of about 20 kPa to about 38 kPa, the propane is at a temperature of about 400° C. to about 650° C., and the regenerated catalyst is at a temperature of about 425° C. to about 750° C. when the propane is contacted with the regenerated catalyst;
removing at least a portion of the propylene from the process environment;
contacting the coked catalyst with a purge fluid to remove at least a portion of any residual propane, at least a portion of any residual propylene, or both from the process environment comprising the coked catalyst, wherein the purge fluid is at a temperature of about 120° C. to about 600° C.;
contacting the coked catalyst with an oxygen-containing fluid within the process environment to combust at least a portion of the coke disposed thereon and produce a decoked catalyst, wherein the oxygen-containing fluid is at a temperature of about 500° C. to about 800° C. when the coked catalyst is contacted with the oxygen-containing fluid;
contacting the decoked catalyst with a reducing gas within the process environment to produce a regenerated catalyst;
stopping contact of the reducing gas with the regenerated catalyst within the process environment;
removing at least a portion of any residual reducing gas from the process environment comprising the regenerated catalyst by exposing the process environment to a vacuum for at least 10 seconds after stopping contact of the reducing gas with the regenerated catalyst; and
contacting additional propane with the regenerated catalyst within the process environment to produce additional propylene and additional coked catalyst comprising coke disposed thereon.

18. The method of claim 17, wherein the reducing gas comprises at least 70 wt % of hydrogen, and wherein the decoked catalyst is contacted with the reducing gas for less than 110 seconds.

19. The method of claim 17, wherein the process environment is at a pressure of about 20 kPa to about 29 kPa when the propane is contacted with the regenerated catalyst.

20. The method of claim 17, further comprising contacting the coked catalyst with a hydrocarbon fuel within the process environment when the cocked catalyst is contacted with the oxygen-containing fluid, wherein the coked catalyst is contacted with the hydrocarbon fuel for at least 77% of the time the coked catalyst is contacted with the oxygen-containing fluid.
